(19) United States
(12) Patent Application Publication
(10) Pub. No.: US 2012/0174324 A1
(43) Pub. Date: Jul. 12, 2012

(54) DYES FOR POLYMER COLORATION, THEIR PREPARATION AND THEIR USE

(75) Inventors: Roxana Barbieru, Singapore (SG); Werner Russ, Wiesbaden-Nordenstadt (DE); Jürgen Kühlwein, Heusenstamm (DE); Carsten Harfmann, Frankfurt am Main (DE)

(73) Assignee: DYSTAR COLOURS DEUTSCHLAND GMBH, Frankfurt am Main (DE)

(21) Appl. No.: 13/263,787

(22) PCT Filed: Apr. 15, 2010
(86) PCT No.: PCT/EP10/54953

§ 371 Date: Mar. 30, 2012

(30) Foreign Application Priority Data
Apr. 22, 2009 (DE) 10 2009 002 563.4

(51) Int. Cl. D06P 1/00; C07D 471/04

(52) U.S. Cl. 8/636; 544/245

(57) ABSTRACT

The present invention relates to a dye of formula (I) where R¹, R², X, Y and t are each as defined in claim 1, to its preparation and to its use.

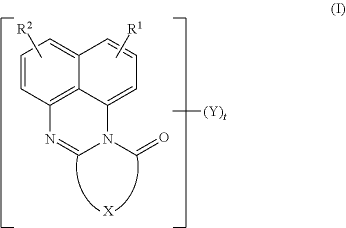

Polymers can be colored with dyes in various ways. One way is mass coloration of polymers whereby for example a pigment or a dye is mixed with the polymer and the polymer is melted to transport the dye into the polymer matrix. Other processes involve the polymer being colored, or to be more precise dyed, by the dyes diffusing into the polymer from a solution or dispersion, examples being the dyeing of polymeric fibers composed of polyester, polyacrylonitrile, polyurethane, cellulose or polyamide for example with disperse dyes, cationic dyes, acid dyes, metallized dyes or reactive dyes. The use of reactive dyes results in a covalent bond being formed between the dye and the substrate, conferring particularly high fastnesses on the dyeings/colorations. Another way to color a polymer is to add the dye to the polymer's monomers or oligomers, before the polymer is formed or as it is being formed. Dyes capable of forming covalent bonds with the polymer scaffold may likewise result in colorations of high fastness being obtained. For this, the dyes used, or to be more precise their chromophores, have to be sufficiently stable under the conditions of the polymerization.

Commercially available pigments when used in mass coloration of polymers do give colored polymers of predominantly high fastnesses, but the colorations are dull, i.e., lack transparency. Commercially available dyes for polymers are usually disperse dyes or solvent dyes and produce, when used for the coloration of polymers, colored polymers in which the dye often only has low bleed fastnesses. In addition, many of the known dyes have poor lightfastnesses or low thermal stabilities in polyolefins. Dyes having good bleed fastnesses, good lightfastnesses, good thermal stabilities as well as high saturation and transparency in polyolefins without adversely affecting the properties of the polyolefins used are not known in large numbers.

There is therefore a need for dyes which have the recited properties and thus are useful for the coloration of polyolefins.

It has now been found that phthaloperinones and also 1,8-naphthaloperinones which bear one or more N-fatty alkyl sulfamoyl groups or N-fatty alkylaryl sulfamoyl groups constitute useful dyes for the coloration of polyolefins and other substrates. They have high stability under application conditions, are readily soluble in the polymer and afford highly transparent colorations having very good fastnesses, particularly a high bleed fastness.

DE 24 39 133 A1, DE 24 47 228, DE 195 48 411 A1, WO 98/15533 and DE 3038899 disclose variously substituted phthaloperinones and also naphthaloperinone useful for solution dyeing or mass coloration of thermoplastic materials, more particularly polyesters, polyamides or vinyl polymers. Polyolefins were not tested as a substrate and no information was published about the fastness profile of the dyes. DE 2724951 likewise discloses dyes of the perinone series as suitable dyes for dyeing or printing fiber materials.

The present invention accordingly provides dyes of formula (I)

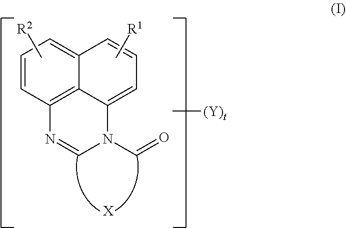

where X represents a group of formula (II) or of formula (III)

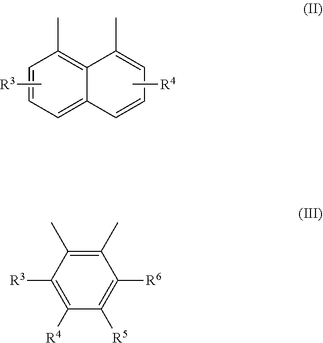

Y represents a group of formula (IV)

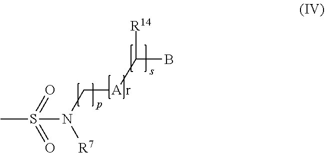

R¹ to R⁶ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₈)-acyloxy, arylcarbonyloxy, (C₂-C₆)-acylamino, arylcarbonylamino, carbamoyl, N-mono-(C₁-C₂₂)-alkylcarbamoyl, N,N-di-(C₁-C₂₂)-alkylcarbamoyl, (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl, mono-(C₁-C₂₂)-alkylaminosulfonyl, di-(C₁-C₂₂)-alkylaminosulfonyl or arylsulfonyl;

R⁷ represents hydrogen, (C₁-C₂₂)-alkyl or phenyl-(C₁-C₂₂)-alkyl;

R¹⁴ represents hydrogen or (C₁-C₆)-alkyl;

A represents a group of formula (V) or of formula (VI)

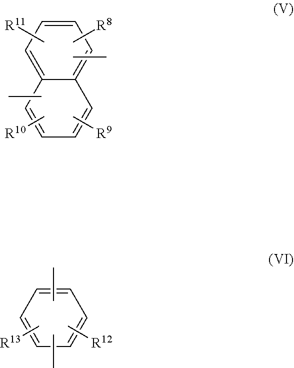

where

R⁸ to R¹³ each independently represent hydrogen, (C₁-C₁₂)-alkyl, trifluoromethyl, halogen, cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio, (C₁-C₆)-alkylsulfonyl, vinyl, hydroxyethyl, mono-(C₁-C₂₂)-alkylaminoethylenesulfonyl, di-(C₁-C₂₂)-alkylaminoethylenesulfonyl, mono-(C₁-C₂₂)-alkylaminosulfonyl or di-(C₁-C₂₂)-alkylaminosulfonyl wherein in each case the (C₁-C₂₂)-alkyl radical may be interrupted by one or more oxygen atoms and/or contain one or more hydroxyl groups;

B represents hydrogen or methyl;

p and s are each independently a number from 0 to 22;

r is 0 or 1; and

t is a number from 1 to 6;

wherein Y is attached to the naphthalene ring bearing R¹ and R², to the group X and/or to an aryl radical which is a constituent part of the R¹ to R⁶ groups, and Y attached to the group of formula (III) represents R³, R⁴, R⁵ and/or R⁶; and

r, s, p and R¹⁴ have identical or different meanings within a molecule of formula (I); and

s is a number from 2 to 22 when X represents a group of formula (III), t is 1, R⁷ represents hydrogen, p is 0 and r is 1; and

excluding dyes of formula (I)
where X represents a group of formula (II), R¹ and R² each represent hydrogen, R³ and R⁴ each represent hydroxyl, t is 1, p and s are each 0, r is 1, A represents a group of formula (VI) and R⁷, R¹², R¹³ and B each represent hydrogen; and where X represents a group of formula (III), R¹ to R⁶ each represent hydrogen, t is 1 and Y represents —SO₂N((C₁-C₆)-alkyl)₂.

In the abovementioned definitions, alkyl groups may be straight chain or branched and be for example methyl, ethyl, n-propyl, isopropyl, n-butyl, isobutyl, hexyl, such as n-hexyl, heptyl, such as n-heptyl, octyl, such as n-octyl and isooctyl, nonyl, such as n-nonyl, decyl, such as n-decyl, dodecyl, such as n-dodecyl, hexadecyl, such as n-hexadecyl and octadecyl, such as n-octadecyl. The sames holds mutatis mutandis for alkoxy, alkylthio and acyl groups.

Cycloalkyl groups are in particular cyclopropyl, cyclobutyl, cyclopentyl, cyclohexyl, cycloheptyl and cyclooctyl.

Halogen is in particular fluorine, chlorine or bromine. Aryl is in particular phenyl or naphthyl.

Phenyl-(C₁-C₂₂)-alkyl R⁷ is in particular phenyl-(C₁-C₆)-alkyl and preferably phenyl-(C₁-C₄)-alkyl. Examples are benzyl and phenethyl.

When R¹⁴ has different meanings within a molecule of formula (I), these different meanings, for example hydrogen and methyl or hydrogen and ethyl, can be randomly distributed, or regions in which R¹⁴ has only one meaning can follow each other in the manner of block polymers.

p is preferably 1, 2, 3 or 4, while t is preferably 1, 2 or 3. s is preferably a number from 0 to 18, but ranges from 2 to 22, 5 to 18, 8 to 18 or 12 to 18 are also possible. In one embodiment of the present invention, r is 0.

Preferred dyes of formula (I) according to the present invention conform to formula (Ia)

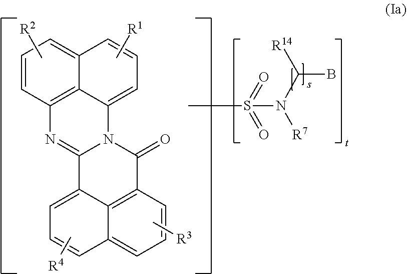

where R¹ to R⁴, R⁷, R¹⁴, B, s and t are each as defined above.

In particularly preferred dyes of formula (Ia) according to the present invention, R¹ to R⁴ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl, mono-(C₁-C₂₂)-alkylaminosulfonyl, di-(C₁-C₂₂)-alkylaminosulfonyl or arylsulfonyl; and

R⁷ represents hydrogen or (C₁-C₂₂)-alkyl; and

R¹⁴, B, s and t are each as defined above.

In very particularly preferred dyes of formula (Ia) according to the present invention R¹ to R⁴ each independently represent hydrogen, halogen, cyano, hydroxyl, (C₁-C₄)-alkoxy or (C₂-C₄)-acyl;

R⁷ represents hydrogen or (C₁-C₁₈)-alkyl;

R¹⁴ represents hydrogen, methyl or ethyl;

B represents hydrogen or methyl;

s is a number from 0 to 17; and

t is a number from 1 to 3.

In the recited very particularly preferred dyes of formula (Ia) it is more particularly R¹ and R² which each represent hydrogen and one of R³ and R⁴ which represents hydrogen while the other represents hydrogen, acetyl, methoxy, chlorine, mono-(C₁-C₈)-alkylaminosulfonyl or di-(C₁-C₈)-alkylaminosulfonyl.

Examples of dyes of formula (Ia) according to the present invention are the dyes of formulae (Ia1) to (Ia41).

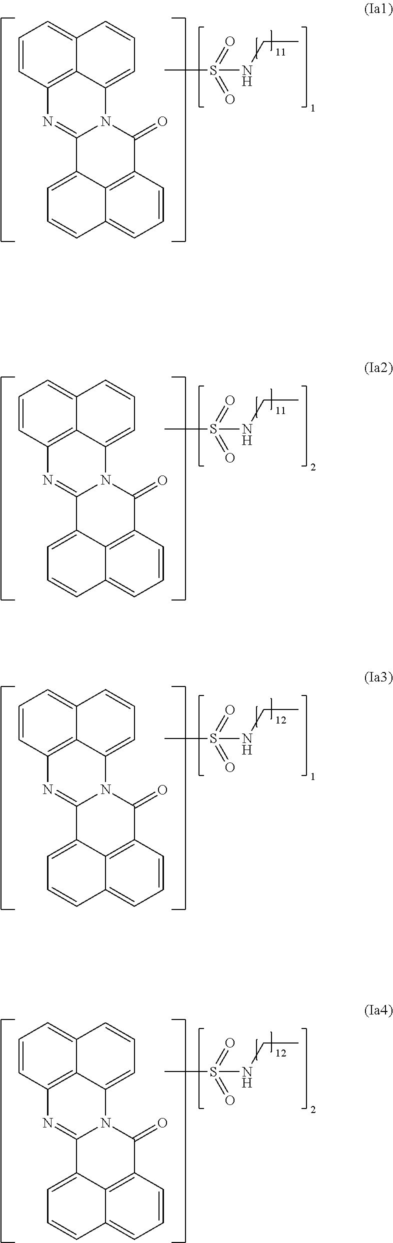

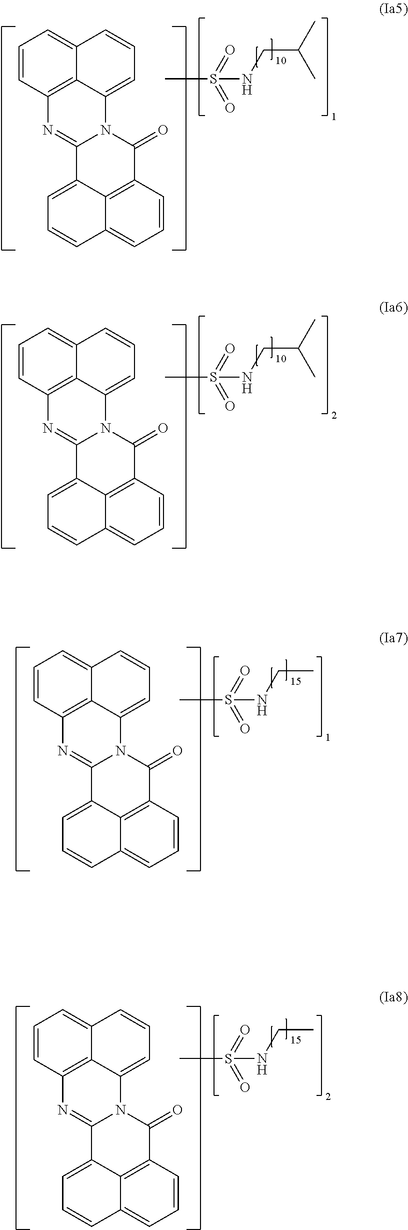

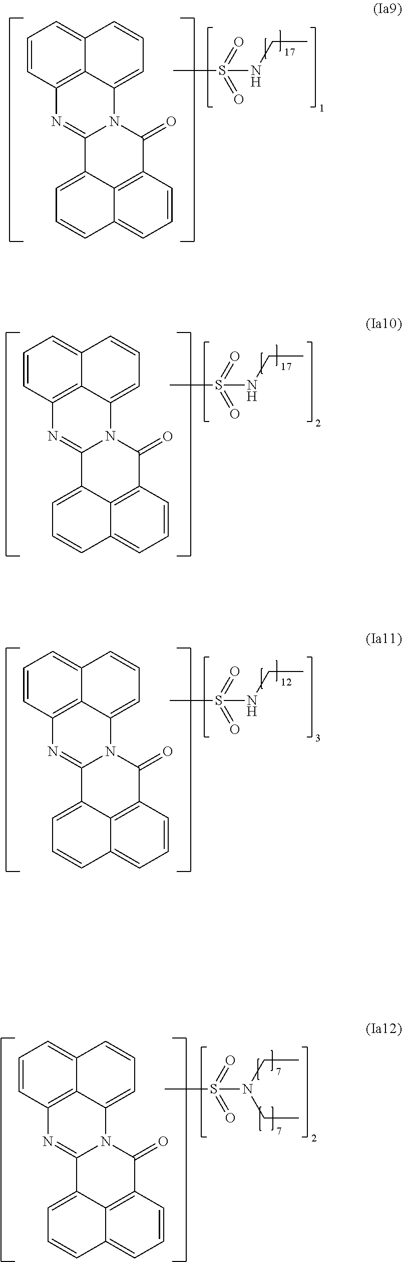

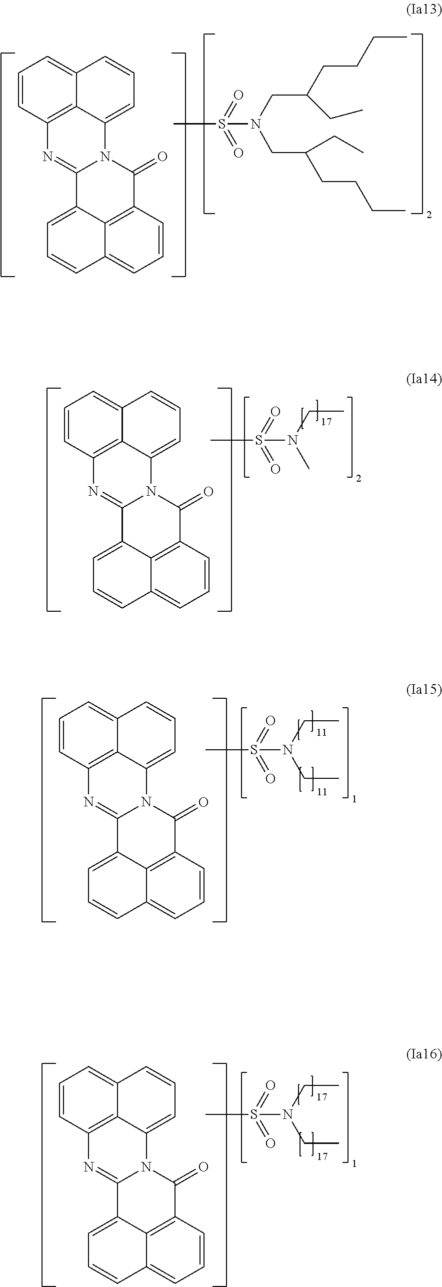

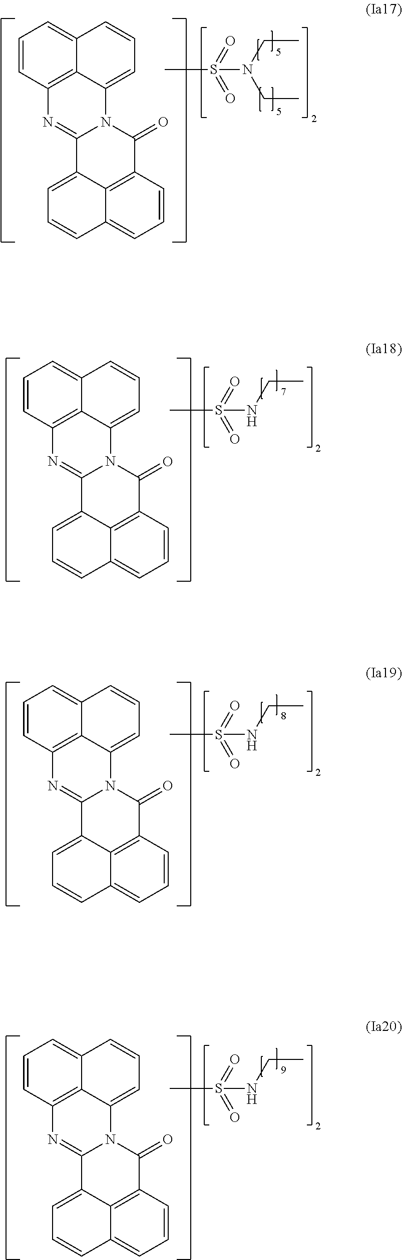

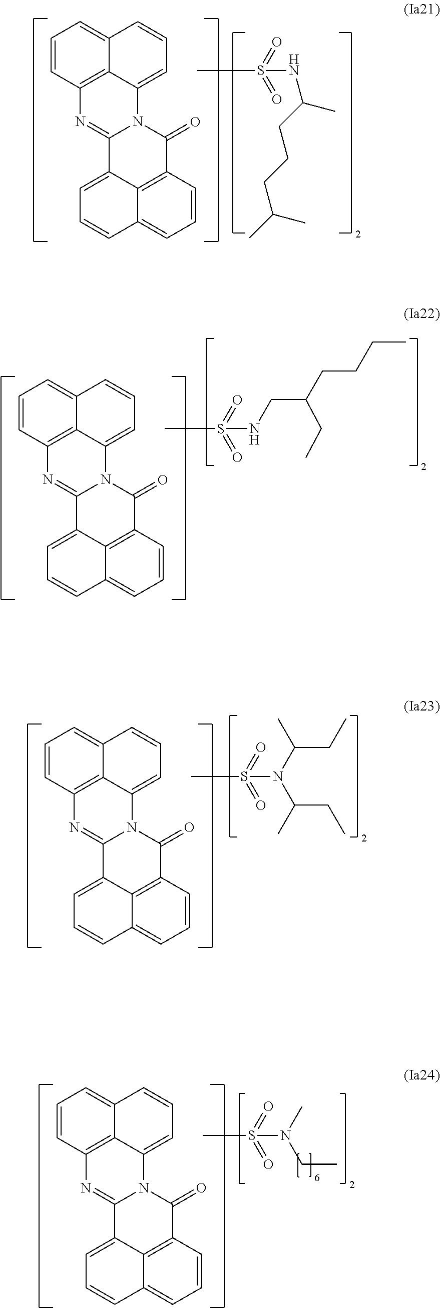

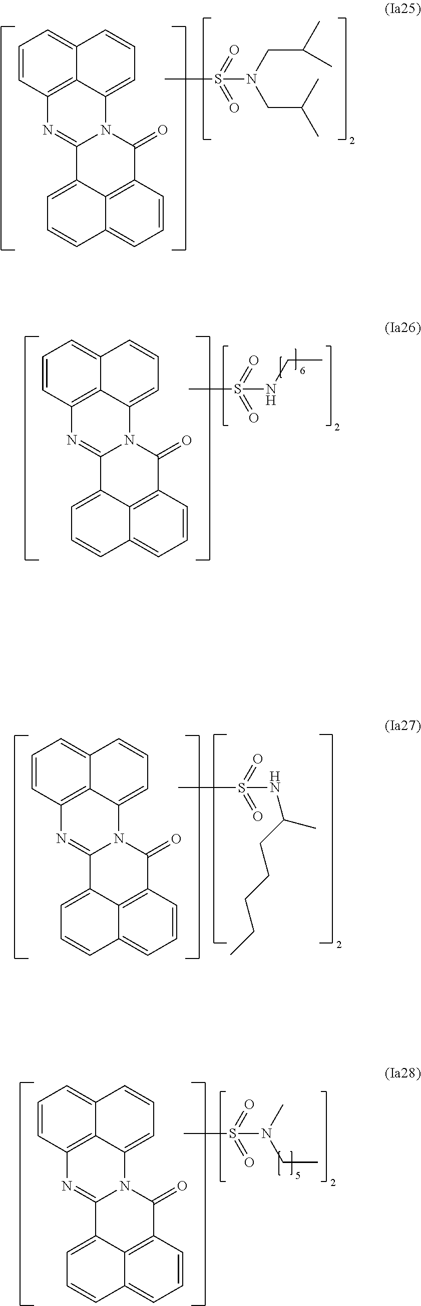

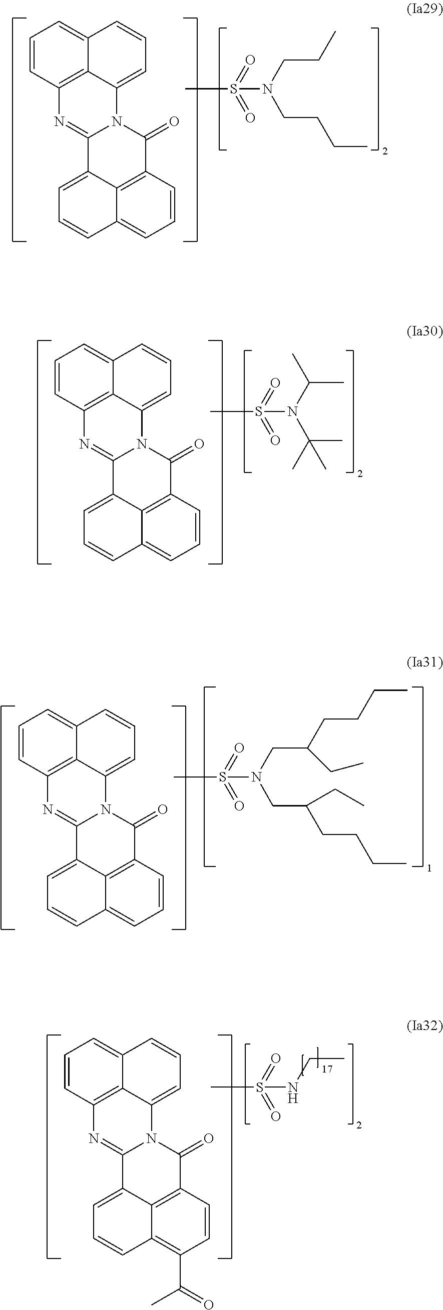

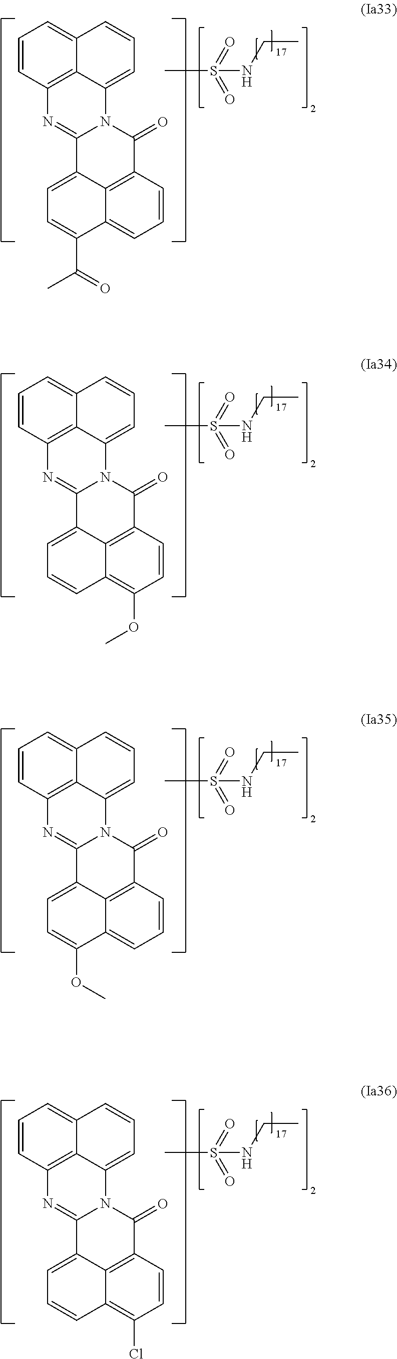

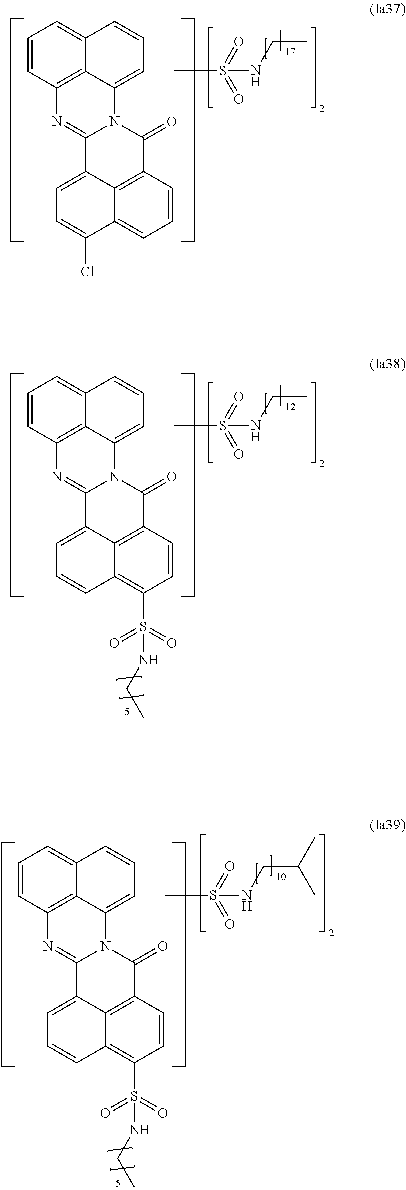

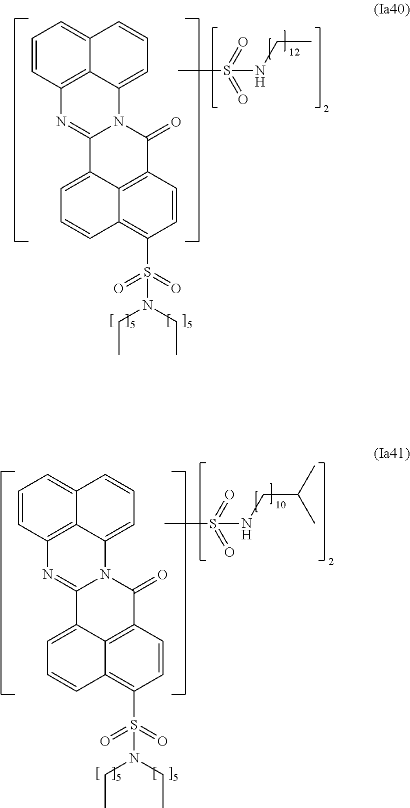

Further preferred dyes of formula (I) according to the present invention conform to formula (Ib)

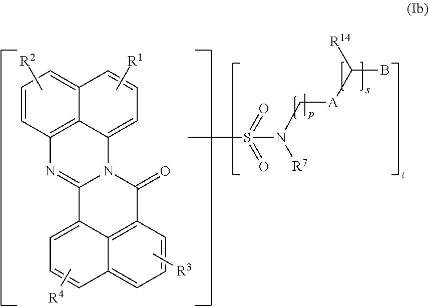

where R¹ to R⁴, R⁷, R¹⁴, A, B, s and t are each as defined above and p is a number from 1 to 22.

In particularly preferred dyes of formula (Ib) according to the present invention, R¹ to R⁴ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, aryl carbonyl, (C₂-C₆)-acyloxy, arylcarbonyloxy, (C₂-C₆)-acylamino, arylcarbonylamino, carbamoyl, (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl, mono-(C₁-C₂₂)-alkylaminosulfonyl, di-(C₁-C₂₂)-alkylaminosulfonyl or arylsulfonyl;

A represents a group of formula (V) or of formula (VI) where R⁸ to R¹³ each independently represent hydrogen, (C₁-C₁₂)-alkyl, halogen, cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio, (C₁-C₆)-alkylsulfonyl mono-(C₁-C₂₂)-alkylaminosulfonyl or di-(C₁-C₂₂)-alkylaminosulfonyl wherein the alkyl radical may be interrupted by one or more oxygen atoms and/or contain one or more hydroxyl groups;

p is a number from 1 to 4; and

s is a number from 0 to 6; and

B, R⁷, R¹⁴ and t are each as defined above.

In very particularly preferred compounds of formula (Ib),

R¹ to R⁴ each independently represent hydrogen, mono-(C₁-C₈)-alkylaminosulfonyl or di-(C₁-C₈)-alkylaminosulfonyl;

A represents a group of formula (V) or formula (VI) where R⁸ to R¹³ each independently represent hydrogen, (C₁-C₆)-alkyl, trifluoromethyl, phenyl, halogen, cyano, nitro, hydroxyl, (C₁-C₄)-alkoxy, (C₂-C₄)-acyl or mono-(C₁-C₁₈)-alkylaminosulfonyl or di-(C₁-C₁₈)-alkylaminosulfonyl wherein the alkyl radical may be interrupted by one or more oxygen atoms and/or contain one or more hydroxyl groups;

B represents hydrogen or methyl;

R⁷ represents hydrogen, (C₁-C₆)-alkyl or phenyl-(C₁-C₆)-alkyl;

R¹⁴ represents hydrogen or (C₁-C₄)-alkyl;

p is a number from 1 to 4;

s is a number from 0 to 4; and

t is 1 or 2.

In the recited very particularly preferred dyes of formula (Ib) it is more particularly R¹ to R³ which each represent hydrogen and R⁴ represents hydrogen, mono-(C₁-C₈)-alkylaminosulfonyl or di-(C₁-C₈)-alkylaminosulfonyl.

Examples of dyes of formula (Ib) are the dyes of formulae (Ib1) to (Ib29).

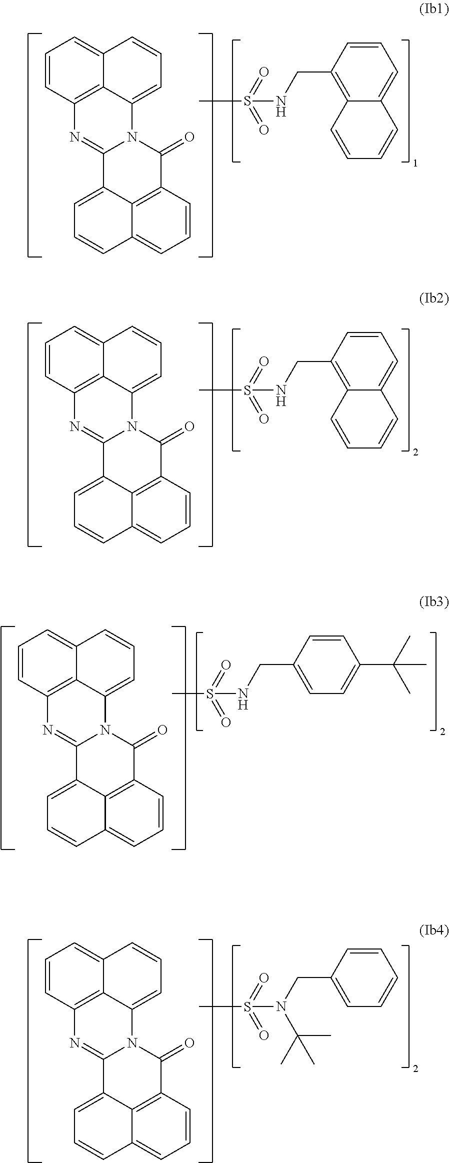

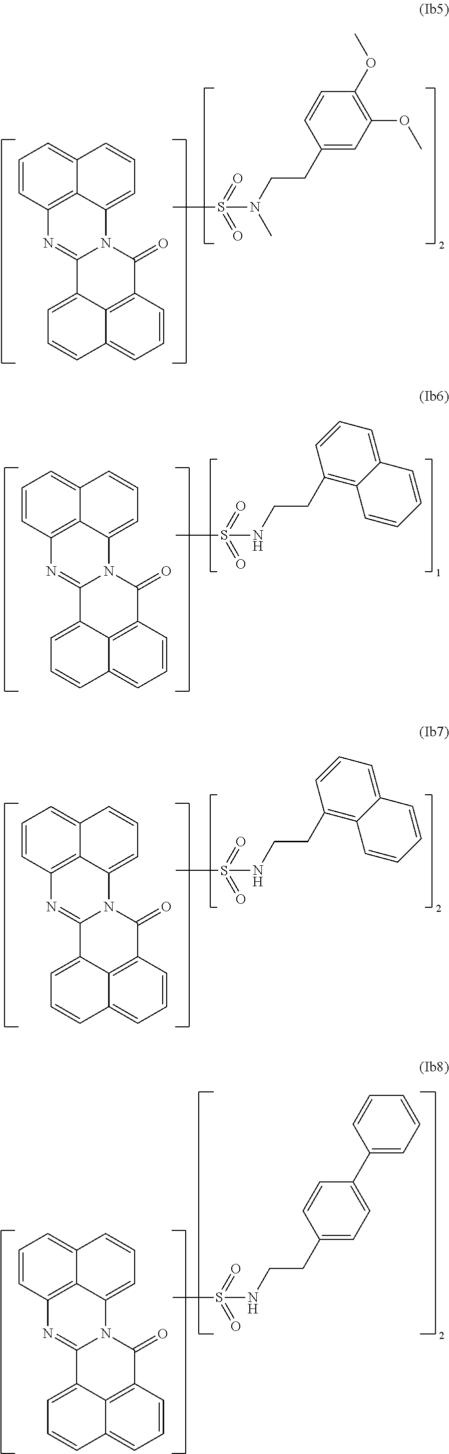

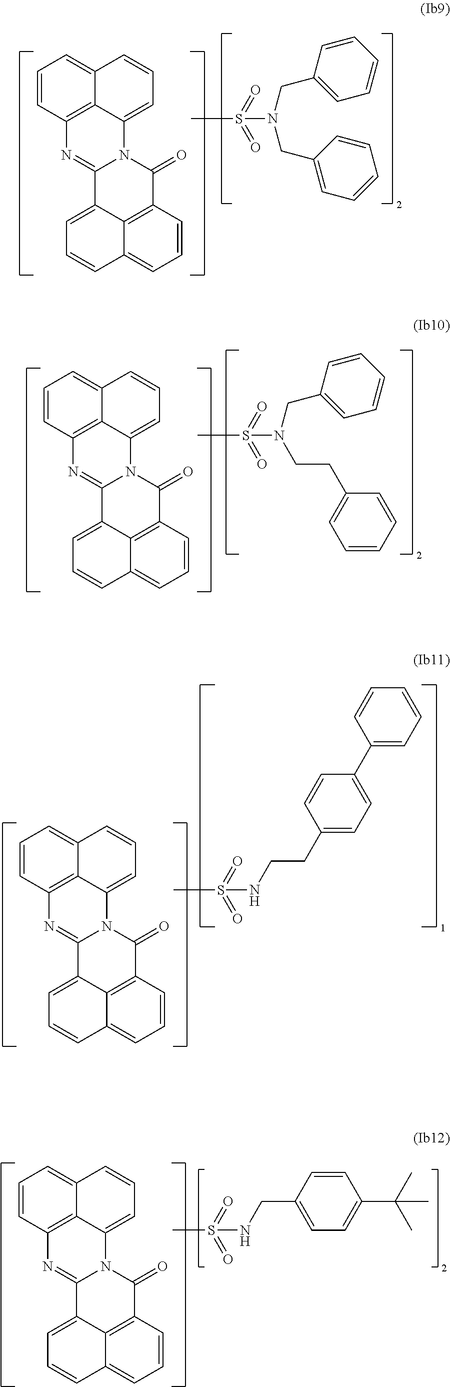

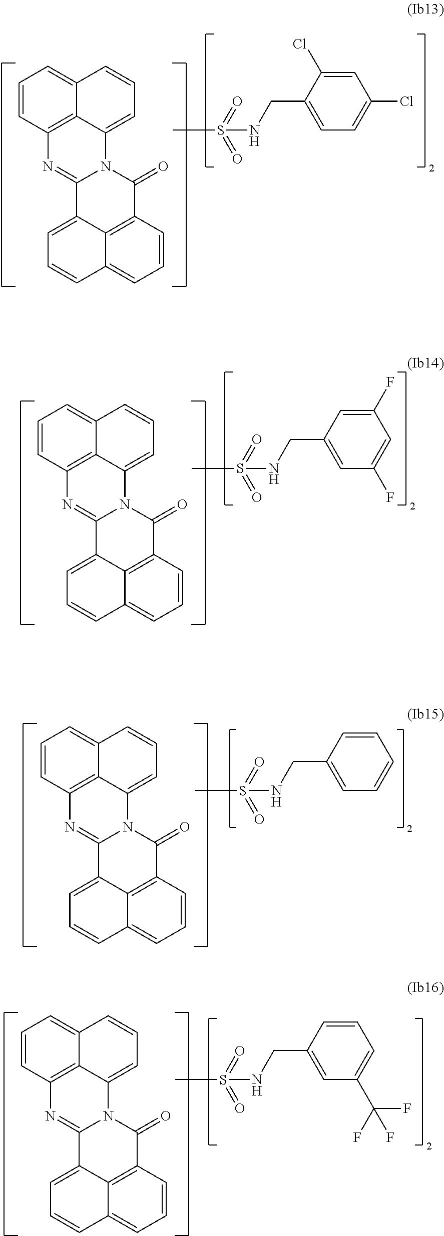

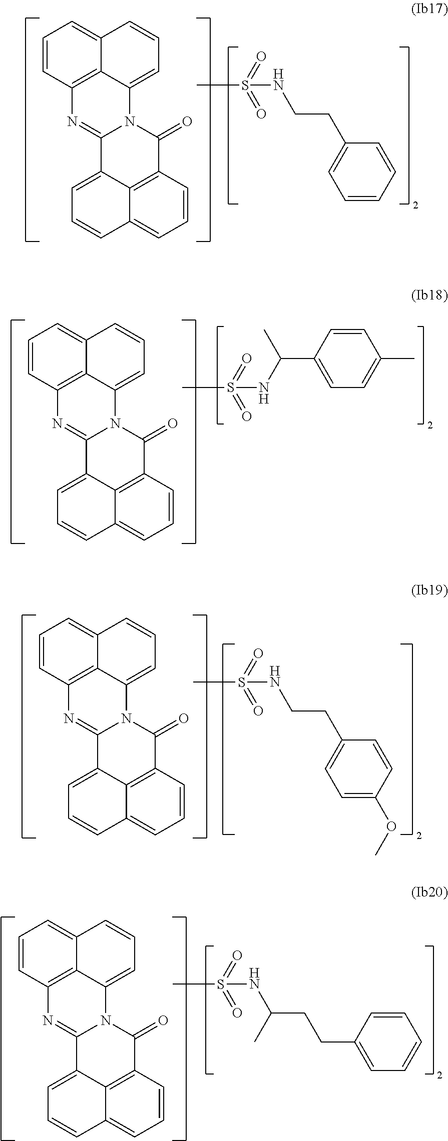

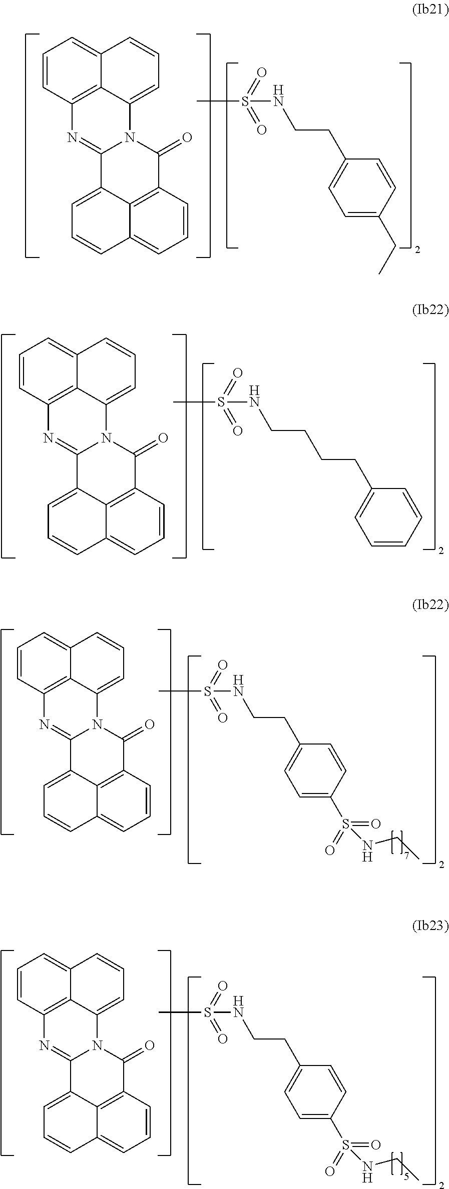

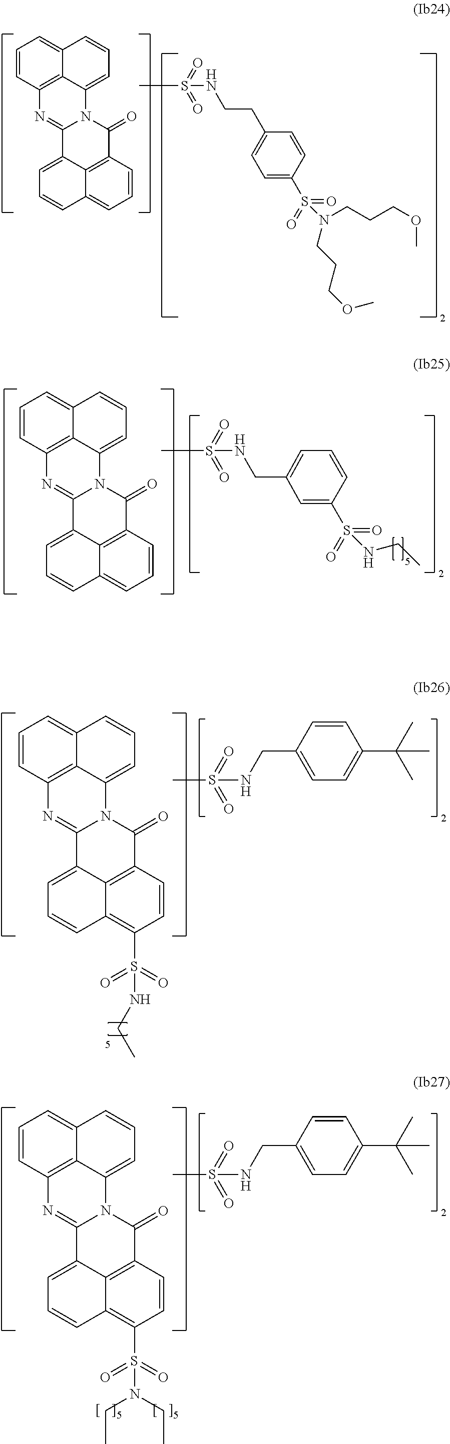

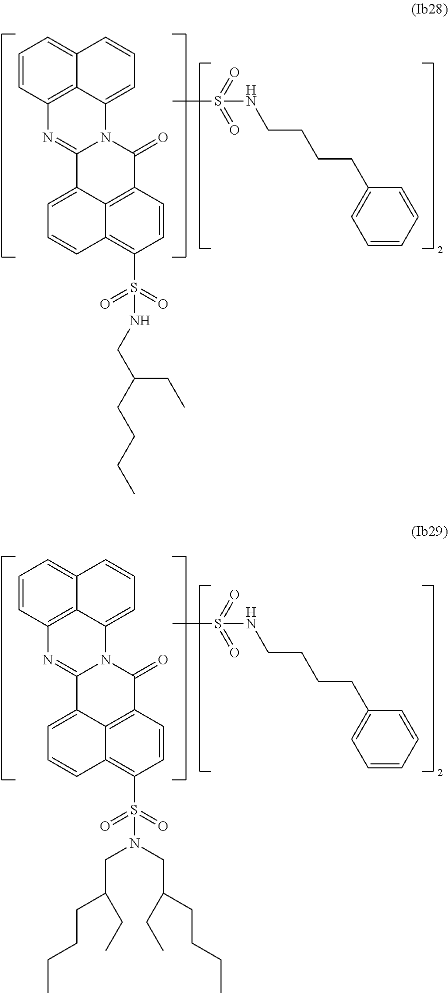

Further preferred dyes of formula (I) according to the present invention conform to formula (Ic)

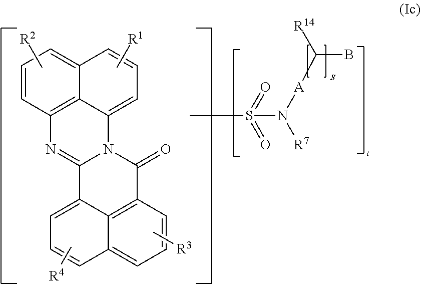

where R¹ to R⁴, R⁷, R¹⁴, A, B, s and t are each as defined above.

In particularly preferred dyes of formula (Ic) according to the present invention,

R¹ to R⁴ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, (C₁-C₆)-alkoxycarbonyl, aryloxycarbonyl, amino, monocyclo-(C₃-C₆)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl, mono-(C₁-C₂₂)-alkylaminosulfonyl, di-(C₁-C₂₂)-alkylaminosulfonyl or arylsulfonyl;

A represents a group of formula (V) or of formula (VI) where

R⁸ to R¹³ each independently represent hydrogen, (C₁-C₁₂)-alkyl, halogen, cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio or (C₁-C₆)-alkylsulfonyl; or mono-(C₁-C₂₂)-alkylaminoethylenesulfonyl, di-(C₁-C₂₂)-alkylaminoethylenesulfonyl, mono-(C₁-C₂₂)-alkylaminosulfonyl or di-(C₁-C₂₂)-alkylaminosulfonyl wherein the (C₁-C₂₂)-alkyl radical may in each case be interrupted by one or more oxygen atoms and/or contain one or more hydroxyl groups; and

R⁷ represents hydrogen, (C₁-C₁₈)-alkyl or phenyl-(C₁-C₄)-alkyl; and

B, R¹⁴, s and t are each as defined above.

In very particularly preferred compounds of formula (Ic)

R¹ to R⁴ each independently represent hydrogen, (C₁-C₄)-alkyl, halogen, cyano, nitro, hydroxyl, (C₁-C₄)-alkoxy, (C₂-C₄)-acyl, (C₂-C₄)-acylamino, carbamoyl, amino, mono-(C₁-C₄)-alkylamino, (C₁-C₄)-alkylthio, mono-(C₁-C₈)-alkylaminosulfonyl, di-(C₁-C₆)-alkylaminosulfonyl or (C₁-C₄)-alkylsulfonyl;

A represents a group of formula (V) or of formula (VI) where R⁸ to R¹³ each independently represent hydrogen, (C₁-C₁₂)-alkyl, halogen; cyano; nitro; hydroxyl; (C₁-C₆)-alkoxy; (C₂-C₆)-acyl; (C₂-C₆)-acylamino; carbamoyl; amino; mono-(C₁-C₁₂)-alkylamino; (C₁-C₆)-alkylthio or (C₁-C₆)-alkylsulfonyl; or mono-(C₁-C₁₈)-alkylaminoethylenesulfonyl, di-(C₁-C₁₈)-alkylaminoethylenesulfonyl, mono-(C₁-C₂₂)-alkylaminosulfonyl or di-(C₁-C₁₈)-alkylaminosulfonyl wherein the (C₁-C₁₈)-alkyl radical may be interrupted by one or more oxygen atoms and/or contain one or more hydroxyl groups;

B represents hydrogen or methyl;

R⁷ represents hydrogen or (C₁-C₆)-alkyl;

R¹⁴ represents hydrogen or (C₁-C₄)-alkyl;

s is a number from 0 to 18; and

t is a number from 1 to 3.

In the recited very particularly preferred dyes of formula (Ic) it is more particularly R¹ to R³ which each represent hydrogen and R⁴ which represents hydrogen, acetyl or methoxy.

Examples of dyes of formula (Ic) are the dyes of formulae (Id) to (Ic29)

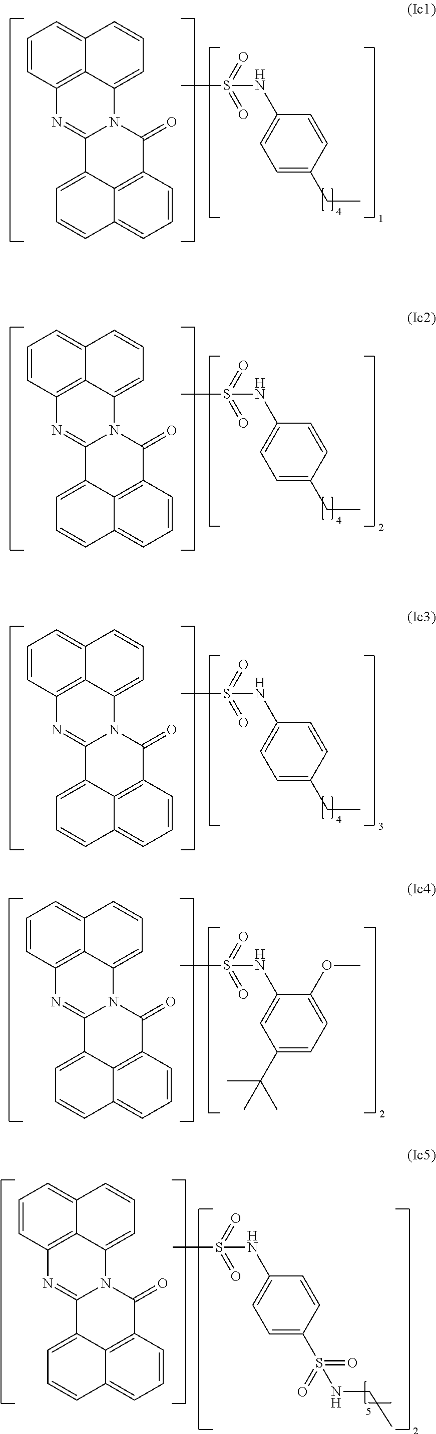

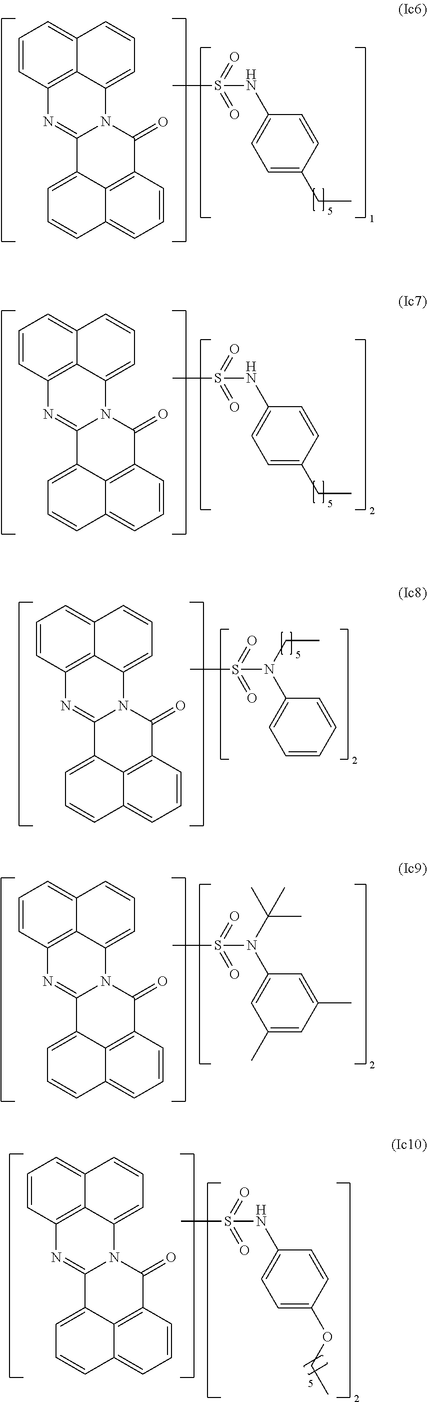

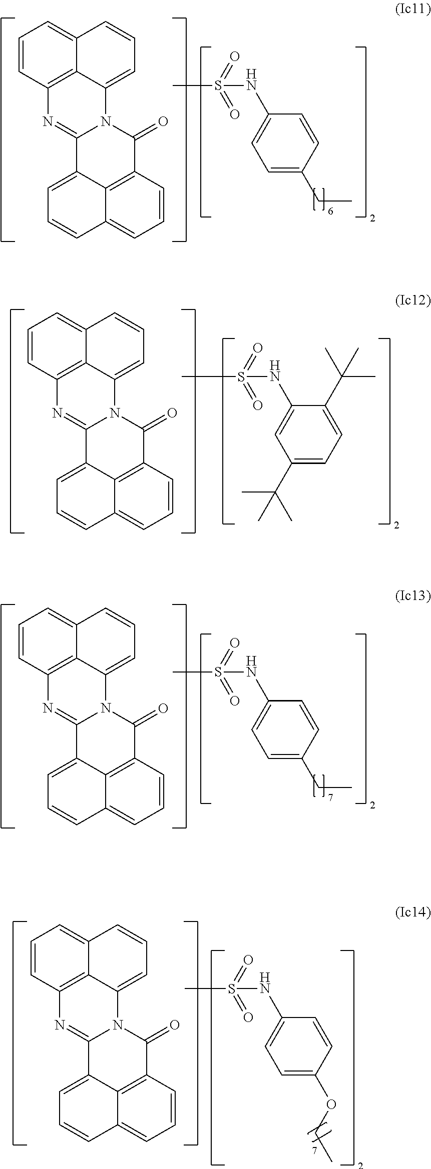

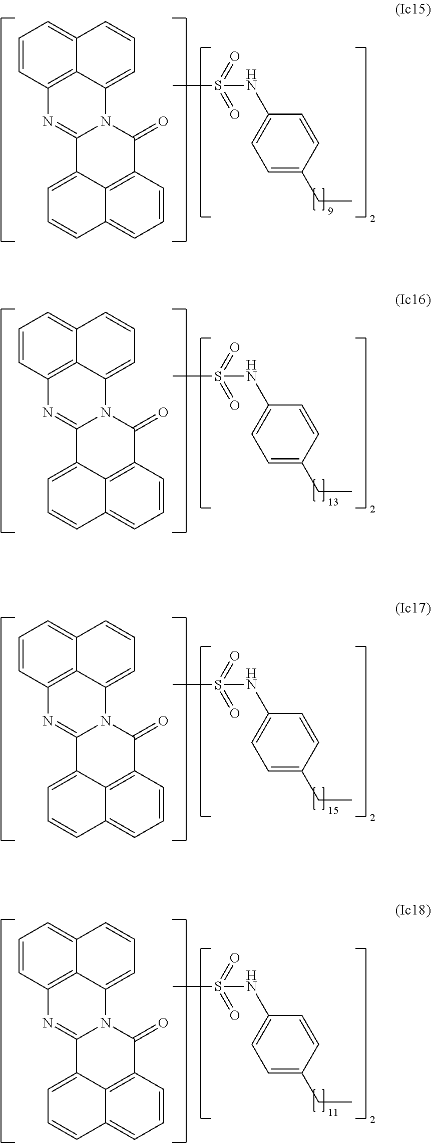

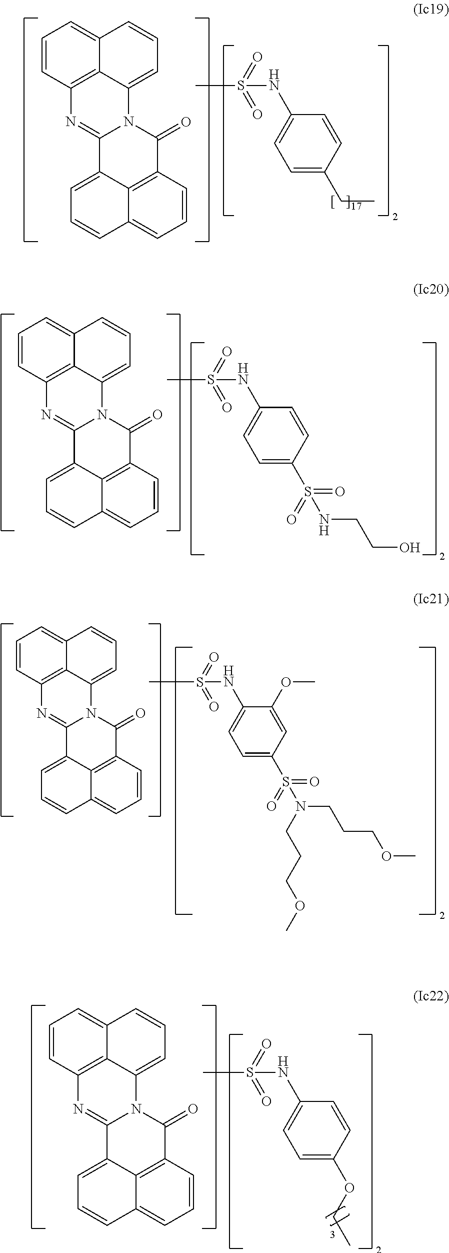

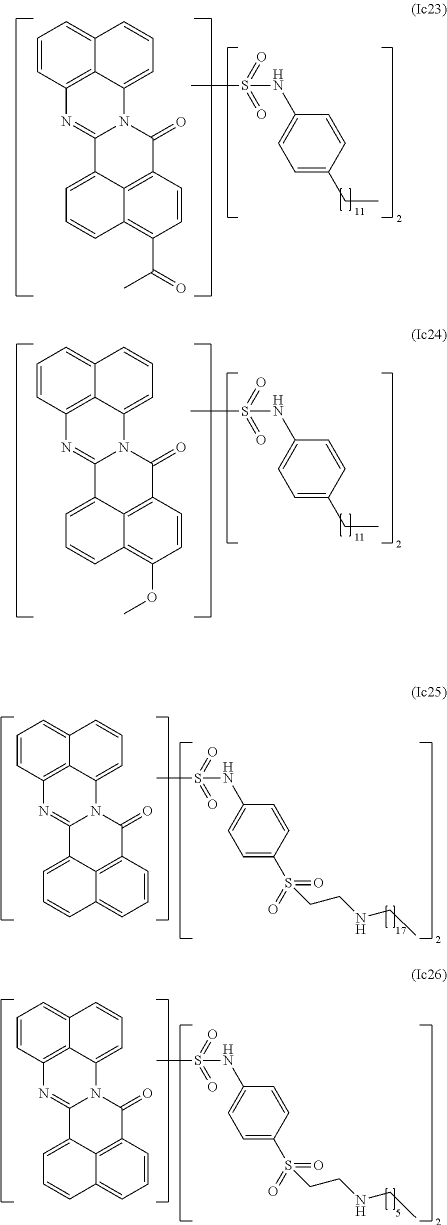

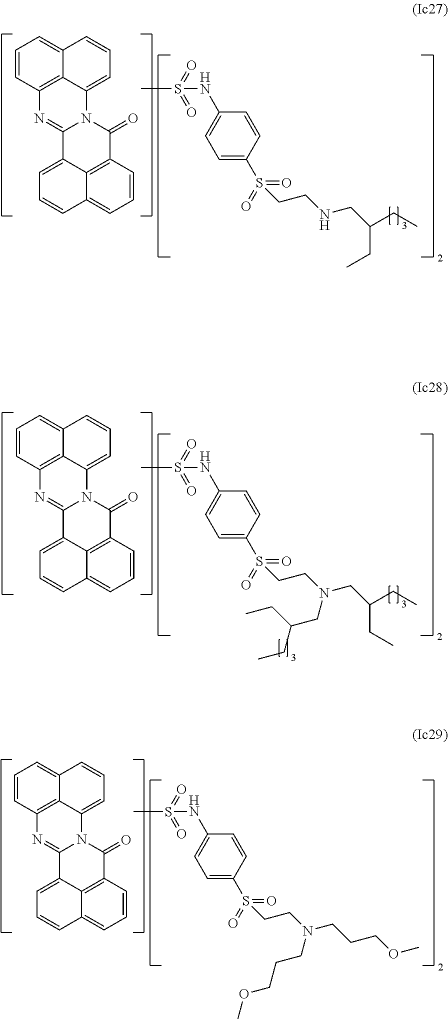

Further preferred dyes of formula (I) according to the present invention conform to formula (Id)

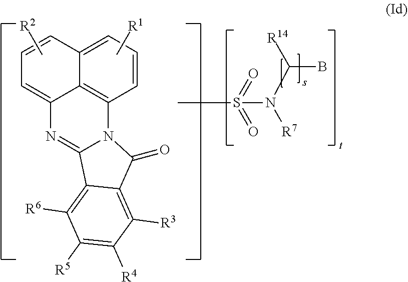

where R¹ to R⁷, R¹⁴, B, s and t are each as defined above.

In particularly preferred dyes of formula (Id) according to the present invention, R¹ to R⁶ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, N-mono-(C₁-C₂₂)-alkylcarbamoyl, N,N-di-(C₁-C₂₂)-alkylcarbamoyl, (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl or arylsulfonyl;

R⁷ represents hydrogen or (C₁-C₂₂)-alkyl; and

B, R¹⁴, s and t are each as defined above.

In very particularly preferred compounds of formula (Id)

R¹ to R⁶ each independently represent hydrogen, halogen; carbamoyl, N-mono-(C₁-C₈)-alkylcarbamoyl or N,N-di-(C₁-C₈)-alkylcarbamoyl;

R⁷ represents hydrogen or (C₁-C₁₈)-alkyl;

R¹⁴ represents hydrogen, methyl or ethyl;

B represents hydrogen or methyl;

p is a number from 0 to 17; and

t is a number from 1 to 3.

In the recited very particularly preferred dyes of formula (Id) it is more particularly R¹ and R² which each represent hydrogen and R³ to R⁶ which each independently represent hydrogen, chlorine, methyl, carbamoyl, N-mono-(C₁-C₈)-alkylcarbamoyl or N,N-di-(C₁-C₈)-alkylcarbamoyl.

Examples of dyes of formula (Id) are the dyes of formulae (Id1) to (Id37)

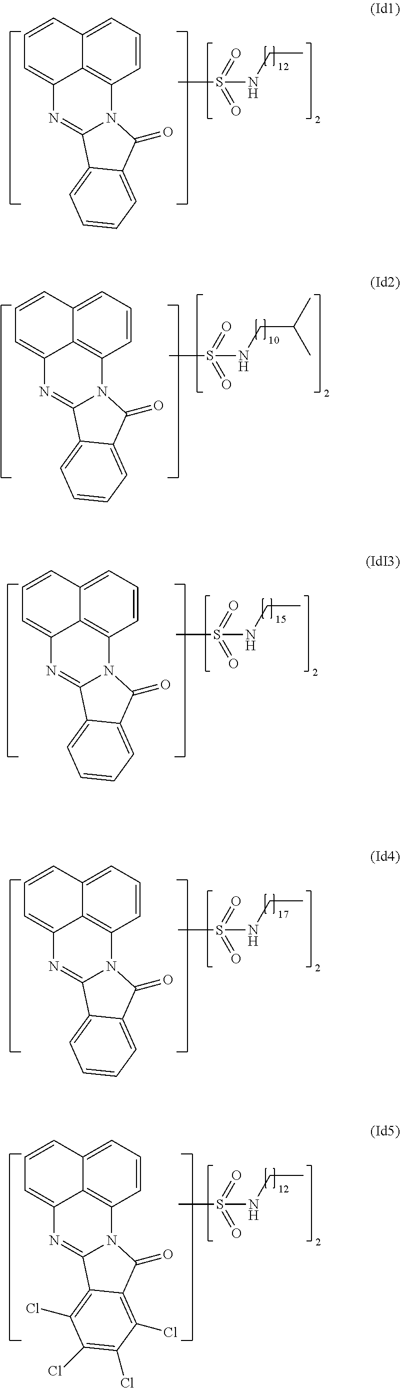

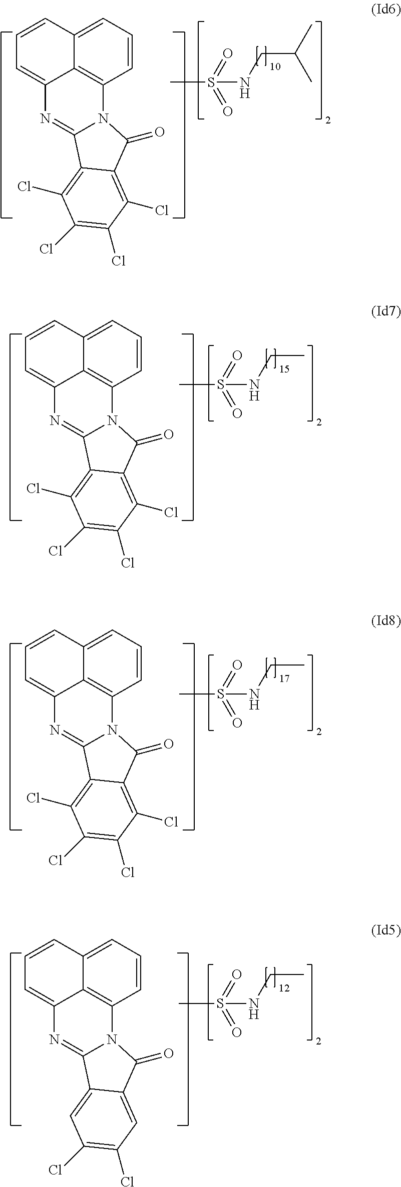

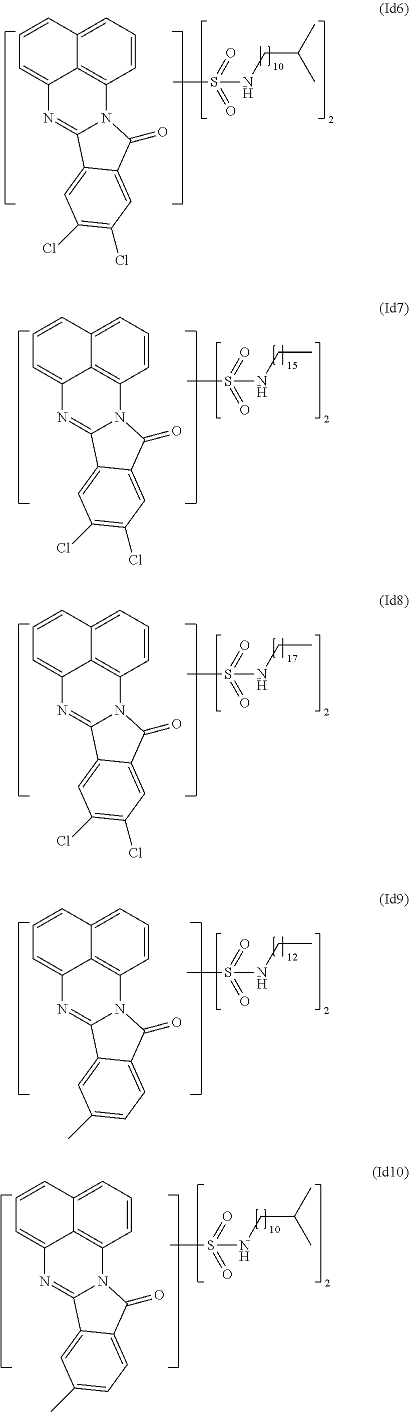

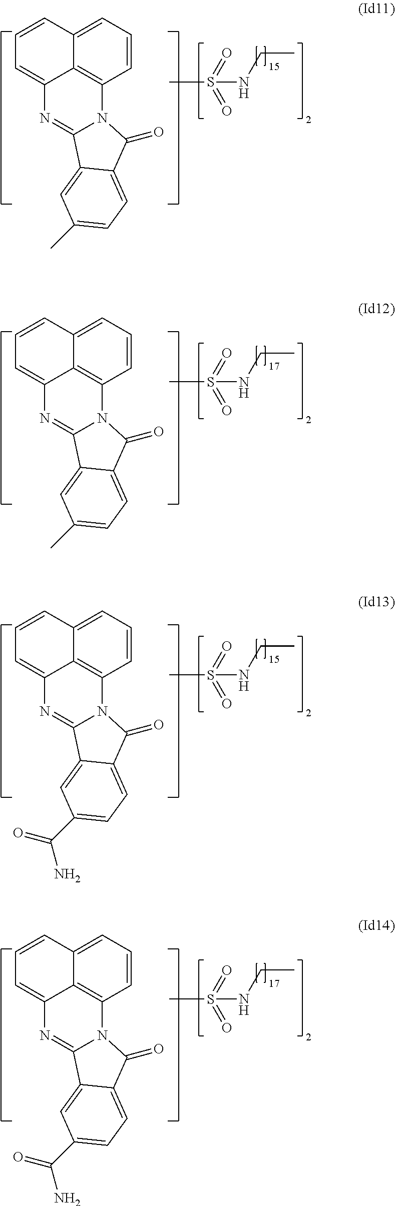

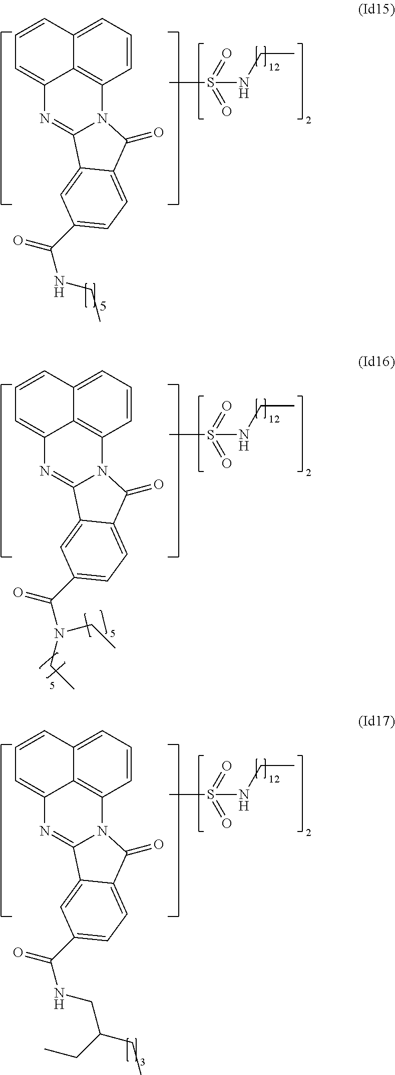

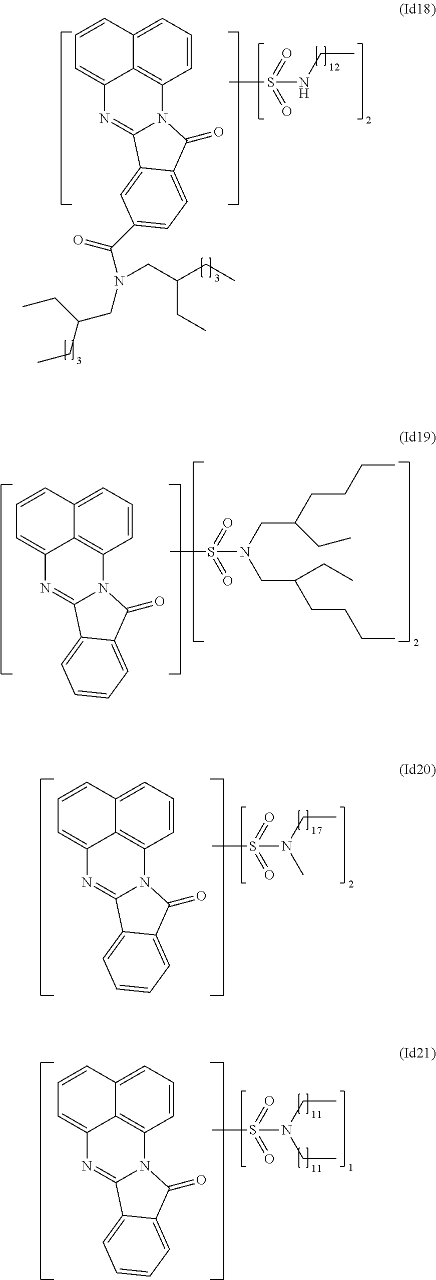

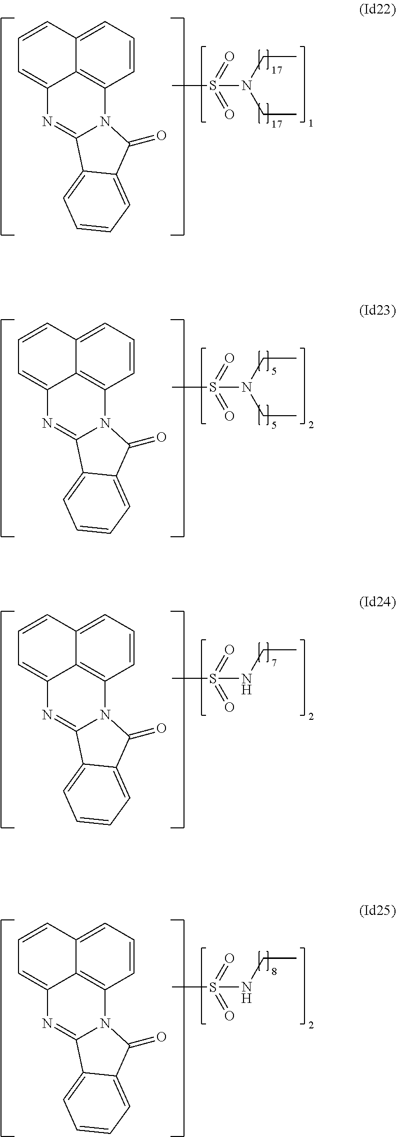

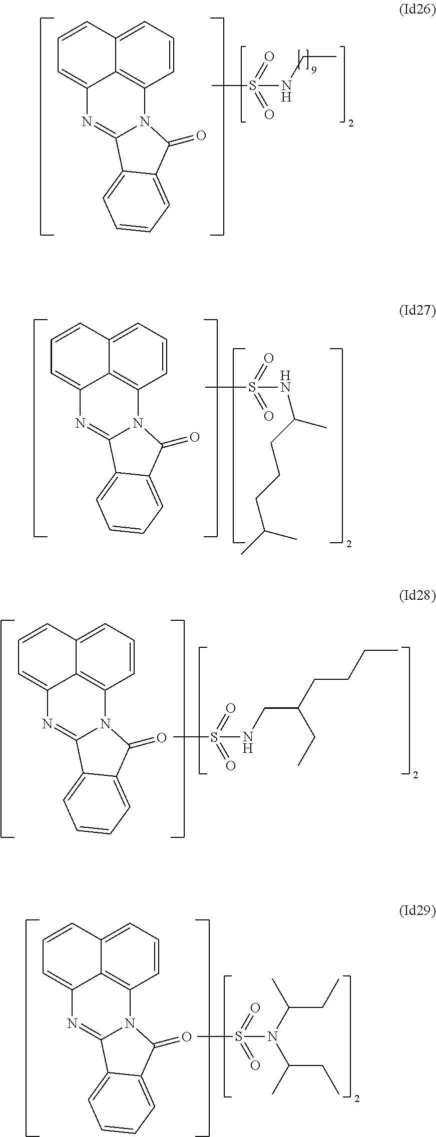

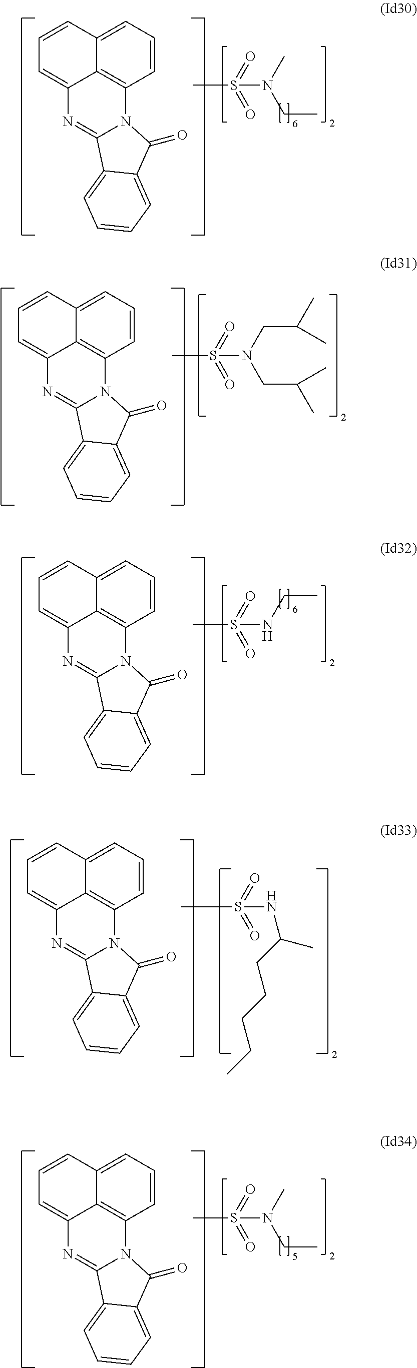

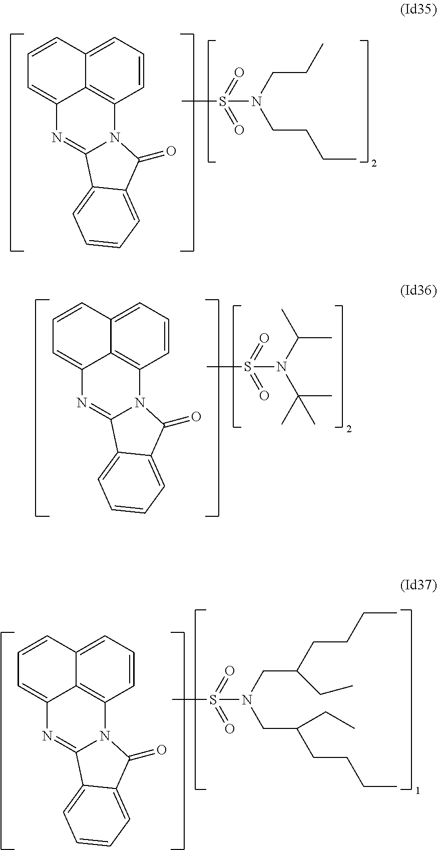

Further preferred dyes of formula (I) according to the present invention conform to formula (Ie)

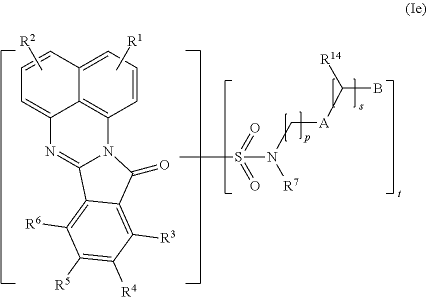

where R¹ to R⁷, R¹⁴, A, B, s and t are each as defined above and p is a number from 1 to 22.

In particularly preferred dyes of formula (Ie) according to the present invention, R¹ to R⁶ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, N-mono-(C₁-C₂₂)-alkylcarbamoyl, N,N-di-(C₁-C₂₂)-alkylcarbamoyl, (C₁-C₆)-alkoxycarbonyl; aryloxycarbonyl, amino, monocyclo-(C₃-C₈)-alkylamino, mono-(C₁-C₆)-alkylamino, di(cyclo)-(C₃-C₈)-alkylamino, monoarylamino, diarylamino, mono-(C₁-C₆)-alkylmonoarylamino, aminothiocarbonylamino, aminocarbonylamino, aminosulfonylamino, alkylthio, arylthio, (C₁-C₆)-alkylsulfonyl, aryloxysulfonyl or arylsulfonyl;

A represents a group of formula (V) or of formula (VI) where

R⁸ to R¹³ each independently represent hydrogen, (C₁-C₁₂)-alkyl, halogen, cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio or (C₁-C₆)-alkylsulfonyl; or mono-(C₁-C₂₂)-alkylaminosulfonyl or di-(C₁-C₂₂)-alkylaminosulfonyl the alkyl radical may be interrupted by one or more oxygen atoms and/or contain one or more hydroxyl groups;

R⁷ represents hydrogen, (C₁-C₁₈)-alkyl or phenyl-(C₁-C₆)-alkyl;

p is a number from 1 to 4; and

s is a number from 0 to 6; and

B, R¹⁴ and t are each as defined above.

In very particularly preferred compounds of formula (Ie)

R¹ to R⁶ each independently represent hydrogen, halogen; carbamoyl, N-mono-(C₁-C₈)-alkylcarbamoyl or N,N-di-(C₁-C₈)-alkylcarbamoyl;

A represents a group of formula (V) or formula (VI) where

R⁸ to R¹³ each independently represent hydrogen, (C₁-C₆)-alkyl, trifluoromethyl, phenyl, halogen, cyano, nitro, hydroxyl, (C₁-C₄)-alkoxy, (C₂-C₄)-acyl or mono-(C₁-C₁₈)-alkylaminosulfonyl or di-(C₁-C₁₈)-alkylaminosulfonyl wherein the (C₁-C₁₈)-alkyl radical may in each case be interrupted by one or more oxygen atoms and/or contain one or more hydroxyl groups;

B represents hydrogen or methyl;

R⁷ represents hydrogen, (C₁-C₆)-alkyl or phenyl-(C₁-C₄)-alkyl;

R¹⁴ represents hydrogen or (C₁-C₄)-alkyl;

p is a number from 1 to 4;

s is a number from 0 to 4; and

t is 1 or 2.

In the recited very particularly preferred dyes of formula (Ie) it is more particularly R¹ and R² which each represent hydrogen and R³ to R⁶ which each independently represent hydrogen, chlorine, methyl, carbamoyl, N-mono-(C₁-C₈)-alkylcarbamoyl or N,N-di-(C₁-C₈)-alkylcarbamoyl.

Examples of dyes of formula (Ie) are the dyes of formulae (Ie1) to (Ie26)

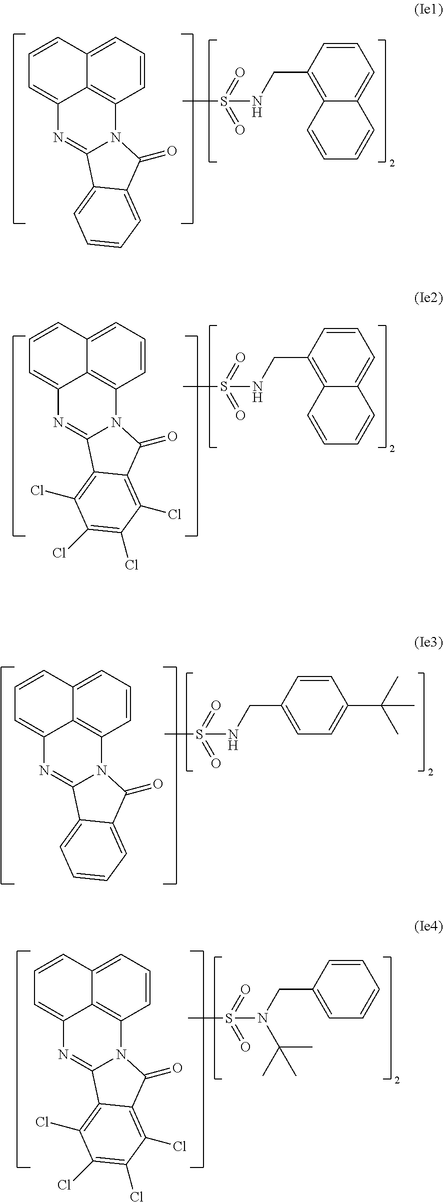

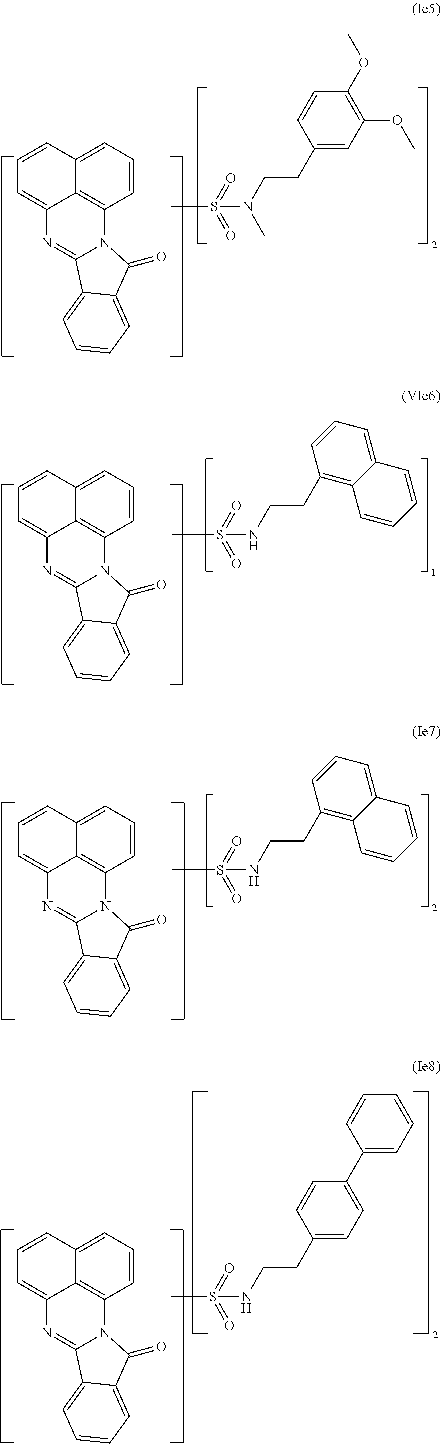

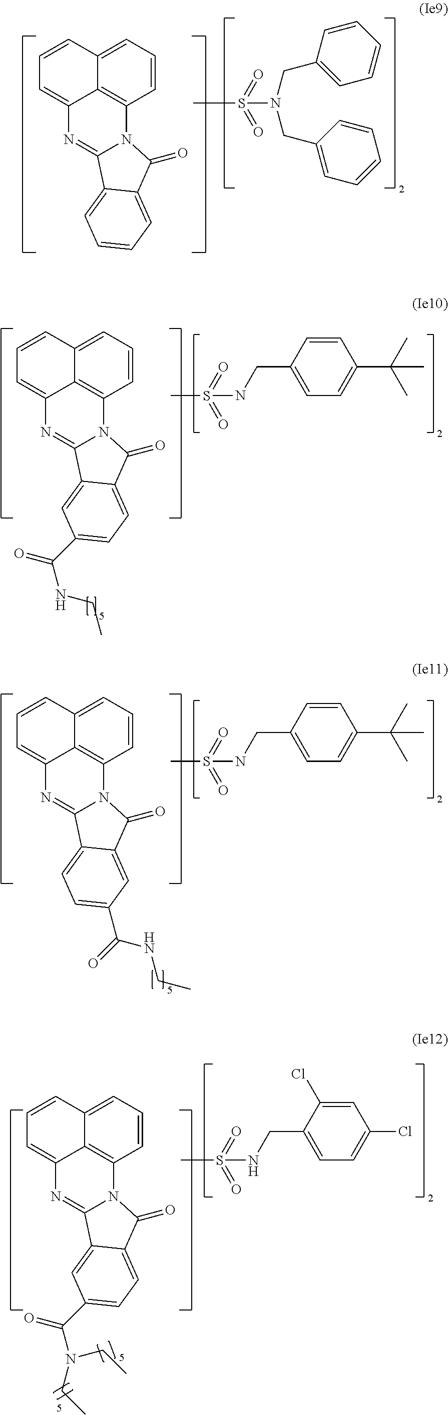

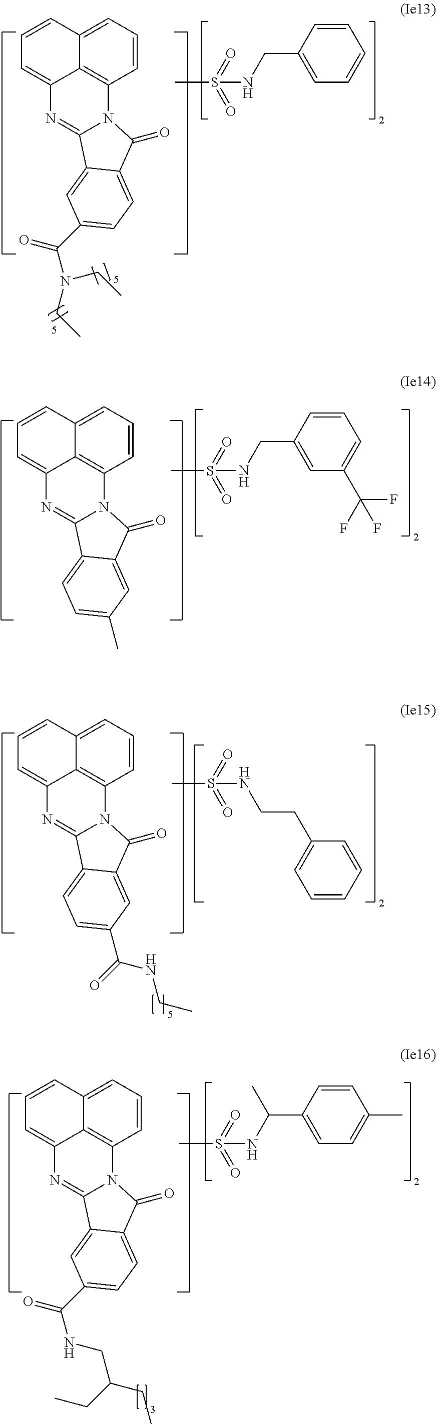

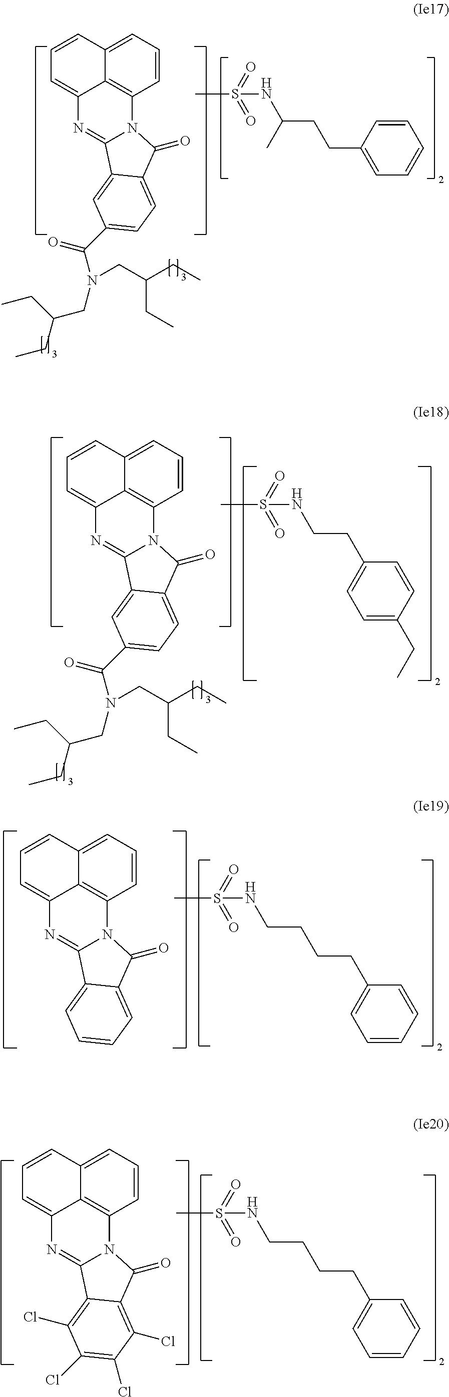

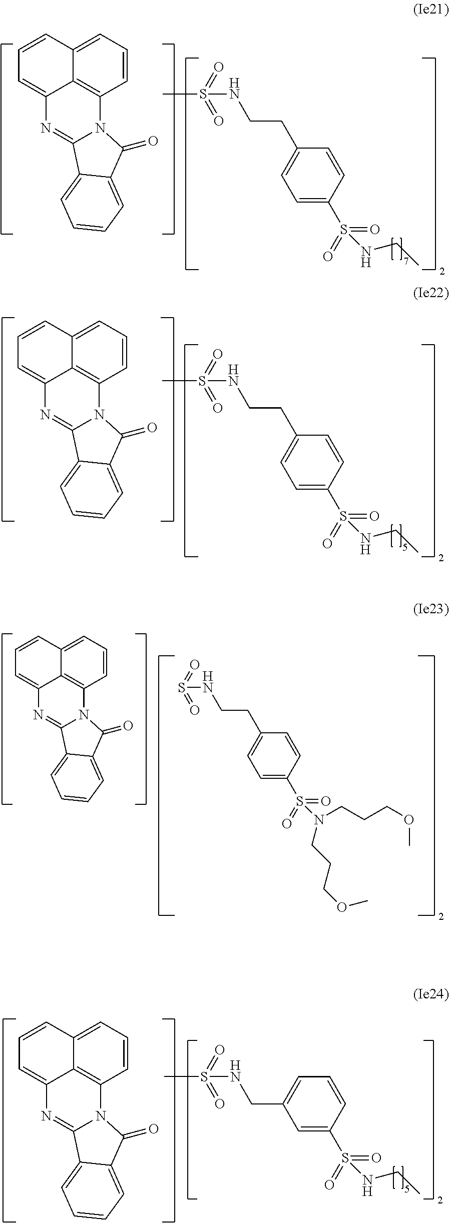

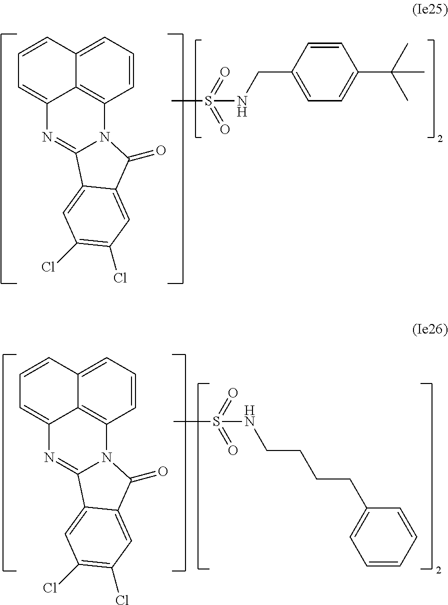

Further preferred dyes of formula (I) according to the present invention conform to formula (If)

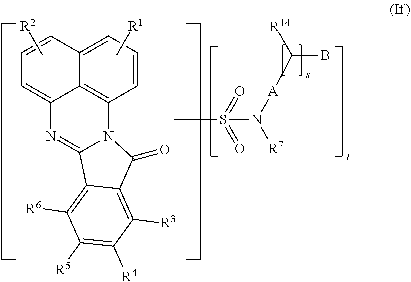

where R¹ to R⁷, R¹⁴, A, B, s and t are each as defined above.

In particularly preferred dyes of formula (If) according to the present invention, R¹ to R⁶ each independently represent hydrogen, (C₁-C₆)-alkyl, trifluoromethyl, cyclo-(C₃-C₆)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl, mono-(C₁-C₂₂)-alkylaminosulfonyl, di-(C₁-C₂₂)-alkylaminosulfonyl or arylsulfonyl;

A represents a group of formula (V) or of formula (VI) where

R⁸ to R¹³ each independently represent hydrogen, (C₁-C₁₂)-alkyl, halogen; cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio, (C₁-C₆)-alkylsulfonyl or mono-(C₁-C₂₂)-alkylaminoethylenesulfonyl, di-(C₁-C₂₂)-alkylaminoethylenesulfonyl, mono-(C₁-C₂₂)-alkylaminosulfonyl or di-(C₁-C₂₂)-alkylaminosulfonyl wherein the (C₁-C₂₂)-alkyl radical may in each case be interrupted by one or more oxygen atoms and/or contain one or more hydroxyl groups;

R⁷ represents hydrogen, (C₁-C₁₈)-alkyl or phenyl-(C₁-C₄)-alkylene; and

B, R¹⁴, s and t are each as defined above.

In very particularly preferred compounds of formula (If)

R¹ to R⁶ each independently represent hydrogen, halogen, carbamoyl, N-mono-(C₁-C₈)-alkylcarbamoyl or N,N-di-(C₁-C₈)-alkylcarbamoyl;

A represents a group of formula (V) or of formula (VI) where

R⁸ to R¹³ each independently represent hydrogen, (C₁-C₁₂)-alkyl, halogen, cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio or (C₁-C₆)-alkylsulfonyl, or represent mono-(C₁-C₁₈)-alkylaminoethylenesulfonyl, di-(C₁-C₁₈)-alkylaminoethylenesulfonyl, mono-(C₁-C₂₂)-alkylaminosulfonyl or di-(C₁-C₁₈)-alkylaminosulfonyl wherein the (C₁-C₁₈)-alkyl radical may be interrupted by one or more oxygen atoms and/or contain one or more hydroxyl groups;

B represents hydrogen or methyl;

R⁷ represents hydrogen or (C₁-C₄)-alkyl;

R¹⁴ represents hydrogen or (C₁-C₄)-alkyl;

s is a number from 0 to 18; and

t is a number from 1 to 3.

In the recited very particularly preferred dyes of formula (If) it is more particularly R¹ and R² which each represent hydrogen and R³ to R⁶ which each independently represent hydrogen or chlorine.

Examples of dyes of formula (If) are the dyes of formulae (If1) to (If30)

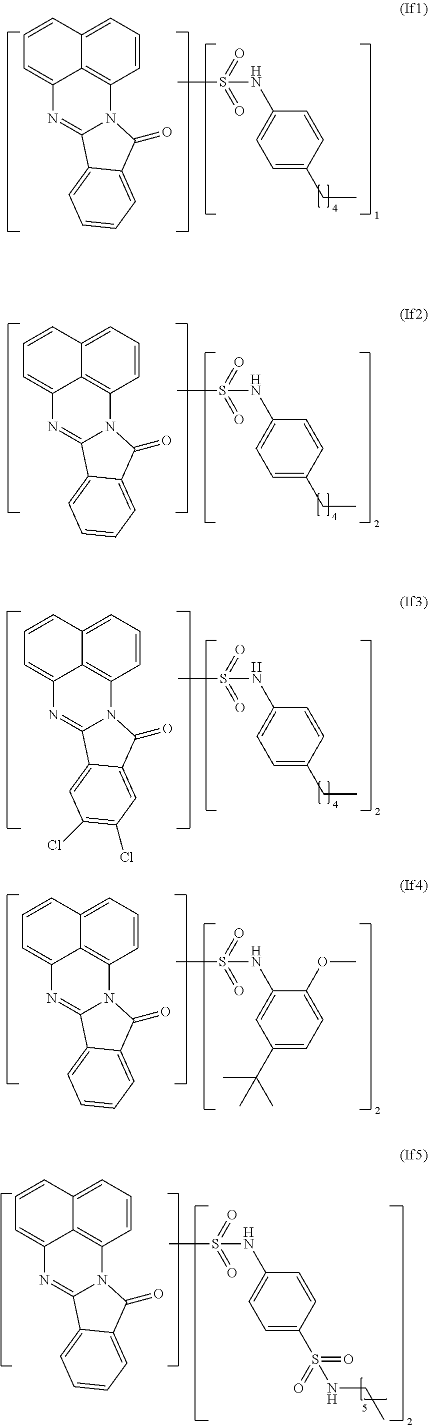

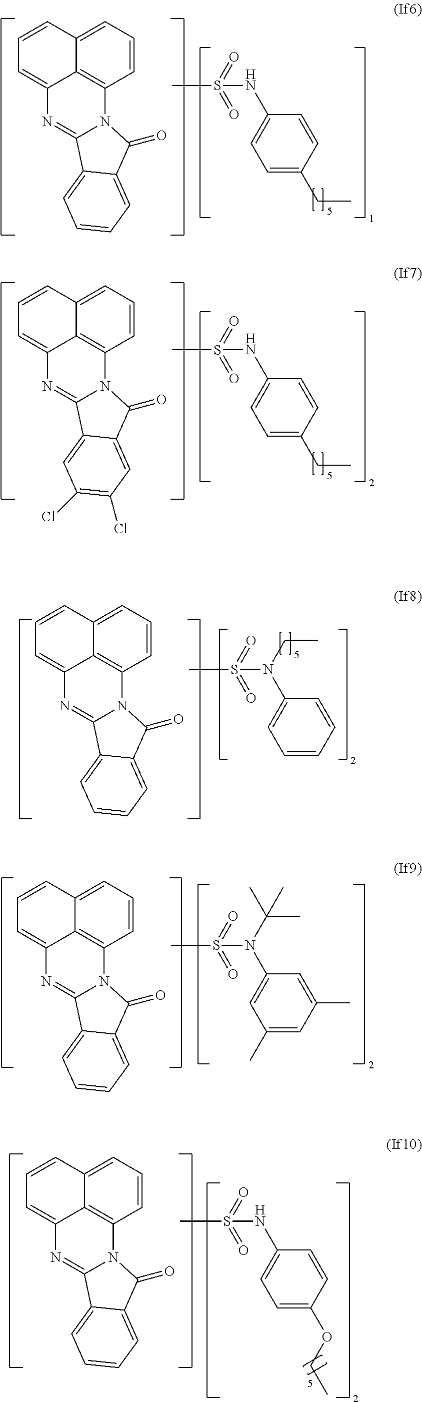

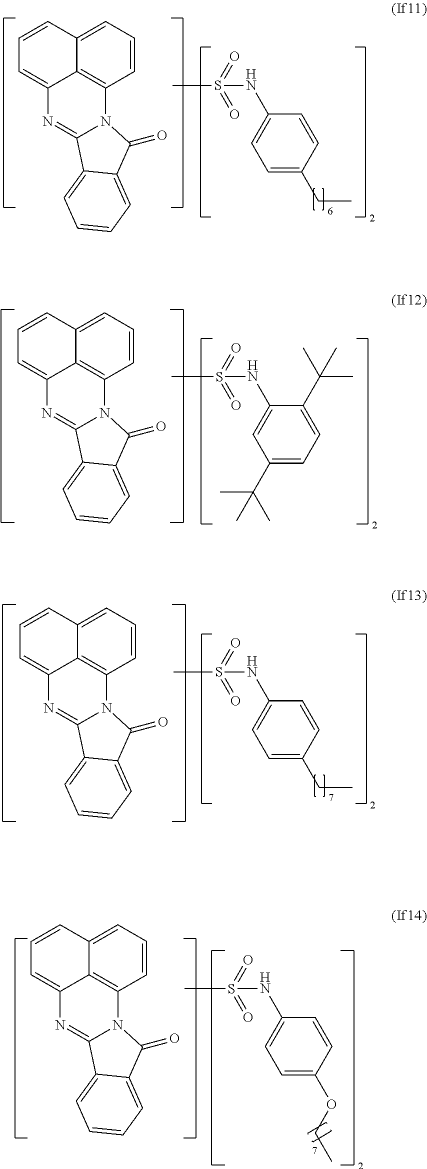

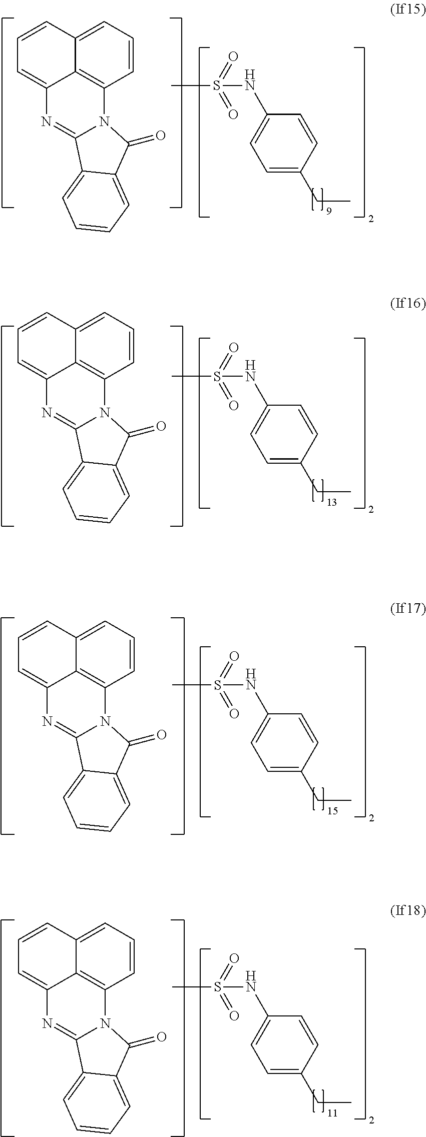

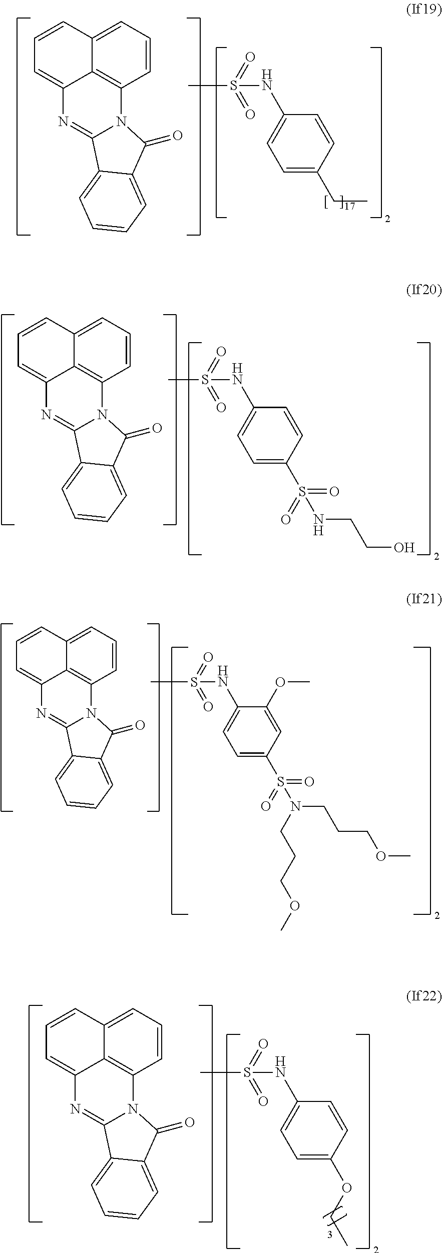

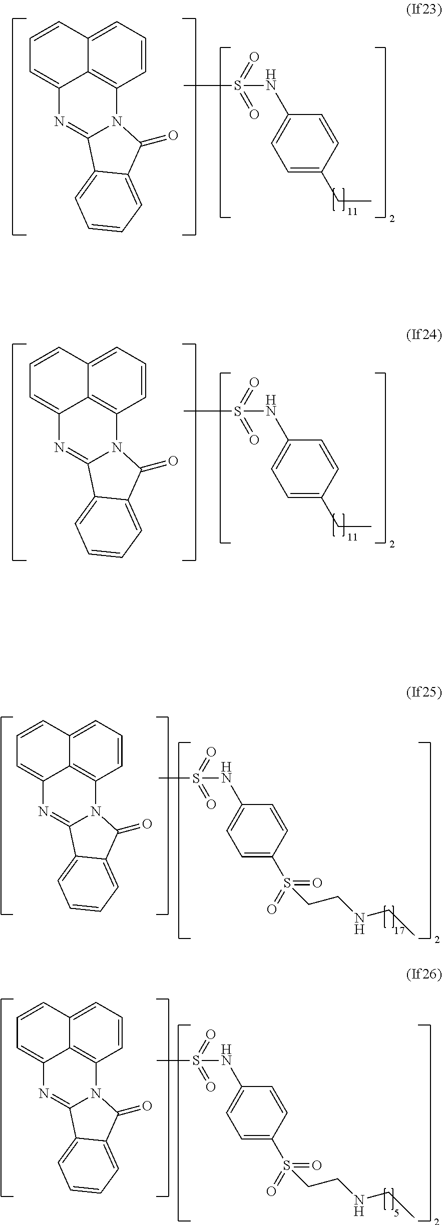

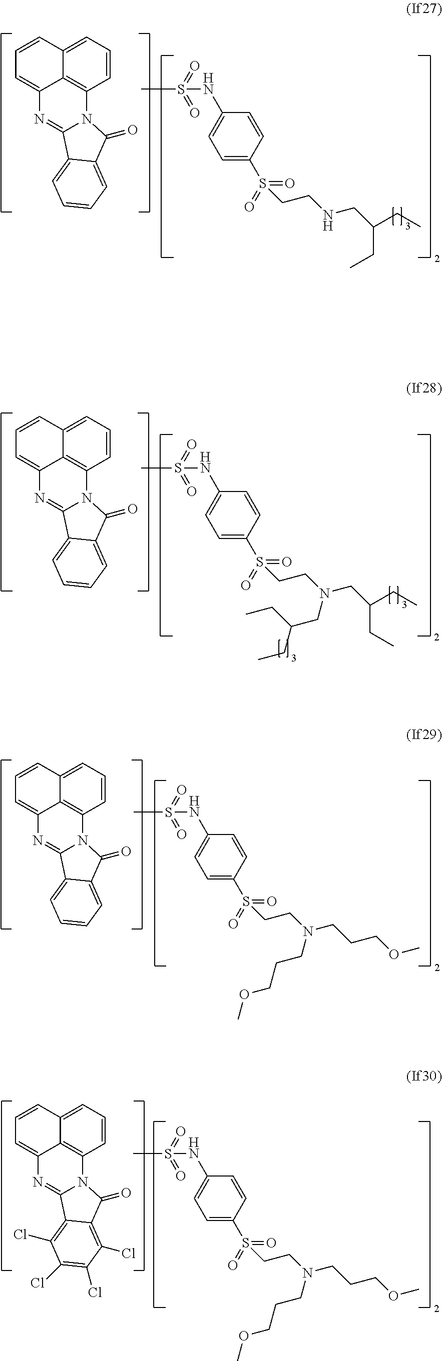

The present invention also provides mixtures between the dyes of formula (I) according to the present invention and also mixtures of one or more dyes of formula (I) with one or more miscible dyes, more particularly pigments or solvent dyes. The respective mixing ratios are not critical and can vary within wide limits.

The present invention further provides a process for preparing a dye of formula (I) according to the present invention, which process comprises reacting a compound of formula (VII)

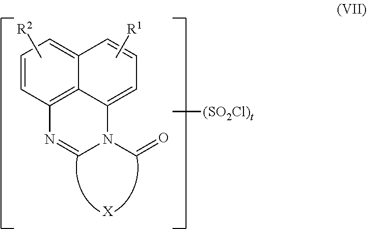

where R¹, R², X and t are each as defined above, with a compound of formula (VIII)

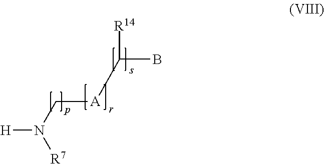

where A, B, R⁷, R¹⁴, p, r and s are each as defined above.

The reacting of the present invention is effected more particularly at temperatures of 25-150° C., more preferably 50-100° C., in the presence or absence of a base such as for example pyridine, piperidine, sodium acetate, potassium carbonate, sodium carbonate, sodium bicarbonate, potassium bicarbonate, sodium methoxide, potassium tert-butoxide or sodium hydroxide. Useful bases further include the compounds of formula (VIII), which can be used in excess.

The reaction medium used is preferably an inert solvent, a mixture of inert solvents, water or a mixture of water and inert solvent. The condensation can also be carried out in the melt.

Particularly preferred solvents are alcohols such as for example n-pentanol, 1-methoxy-2-propanol, 2-ethylhexanol, 2-methyl-1-butanol, isoamyl alcohol, benzyl alcohol, cyclohexanol, glycols and derivatives thereof such as for example ethylene glycol diethyl ether, ethylene glycol monomethyl ether, ethylene glycol monoethyl ether, ethylene glycol monoisopropyl ether, ethylene glycol monobutyl ether, diethylene glycol dimethyl ether, diethylene glycol diethyl ether, diethylene glycol monomethyl ether, ethylene glycol, diethylene glycol monoethyl ether, dipropylene glycol, ethers such as for example dibutyl ether, diisobutyl ether, diisoamyl ether, di-n-amyl ether, or further polar or apolar inert solvents such as for example acetone, ethylbenzene, anisole, N,N-dimethylformamide, N,N-dimethylacetamide, sulfolane, N-methylpyrrolidone, toluene, 1,2-xylene, 1,3-xylene, 1,4-xylene, chlorobenzene, 1,2-dichlorobenzene or mixtures thereof.

The reaction of the present invention can be carried out in solution (in a single phase) or in a reaction system containing two or more liquid phases which are mutually immiscible under the reaction conditions. If they are carried out under multi-phase conditions, phase transfer catalysts can be used. Suitable phase transfer catalysts are the groups of tetraalkylammonium salts, benzyltrialkylammonium salts, tetra-alkylphosphonium salts, benzyltrialkylphosphonium salts and mixtures thereof. For the purposes of the process according to the present invention, ammonium salts are preferable over phosphonium salts and of particular suitability are the tetra-n-butylammonium, tri-n-butylmethylammonium and benzyltriethylammonium salts with the anions chloride, bromide and hydrogensulfate. Very particular preference for use as phase transfer catalysts is given to tetrabutylammonium bromide (Aliquat 100®), tricaprylylmethylammonium chloride (Aliquat 336®), methyltrioctylammonium chlorides (Aliquat 134®), methyltributylammonium chloride (Aliquat 175®), dibenzo-18-crown-6 (Aliplex 186 DB®), benzyltriethylammonium chloride, hexadecyltributylphosphonium bromide and mixtures thereof.

The compounds of formula (VII) are obtainable for example by sulfochlorinating the compounds of formula (IX)

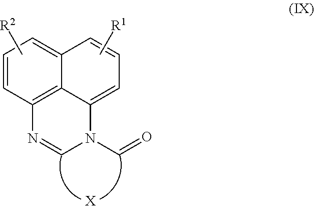

where R¹, R² and X are each as defined above.

The sulfochlorination can be carried out in a conventional manner in chlorosulfonic acid in the presence or absence of thionyl chloride. The sulfochlorination is more particularly effected at a temperature of 25-150° C. and more preferably of 50-130° C. Typically, a mixture of isomers is obtained. The conditions of the reaction are chosen in particular such that t preferably attains values from 1 to 3. Conditions leading to the introduction of two sulfonyl chloride radicals are particularly preferred.

The compounds of formula (IX) are known and commercially available, or are readily obtainable from known starting materials similarly to known methods.

Examples of commercially available compounds of formula (IX) are for example Colour Index Solvent Orange 60, Colour Index Solvent Red 135 and Colour Index Solvent Red 179, and also the compounds marketed by DyStar Textilfarben GmbH under the trade name of ColyPlast® and also the compounds marketed by Lanxess Deutschland GmbH under the trade name of Macrolex®.

The compounds of formula (IX) are obtainable if necessary by condensation of the aromatic anhydrides of formula (X)

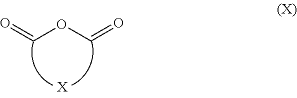

where X is as defined above, or of the corresponding dicarboxylic acids or esters with the compounds of formula (XI)

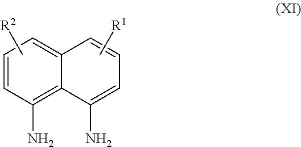

where R¹ and R² are each as defined above.

The reaction medium used for the condensation is preferably an inert solvent, a mixture of inert solvents, an inorganic acid, an organic acid or an aqueous solution thereof. The condensation can also be carried out in the melt. Particularly preferred inert solvents are glycols and derivatives thereof such as for example ethylene glycol diethyl ether, ethylene glycol monomethyl ether, ethylene glycol monoethyl ether, ethylene glycol monoisopropyl ether, ethylene glycol monobutyl ether, diethylene glycol dimethyl ether, diethylene glycol diethyl ether, diethylene glycol monomethyl ether, ethylene glycol, diethylene glycol monoethyl ether, dipropylene glycol, ethers such as for example dibutyl ether, diisobutyl ether, diisoamyl ether, di-n-amyl ether, or further polar or apolar inert solvents such as for example acetone, ethylbenzene, anisole, N,N-dimethylformamide, N,N-dimethylacetamide, sulfolane, N-methylpyrrolidone, toluene, 1,2-xylene, 1,3-xylene, 1,4-xylene, chlorobenzene, 1,2-dichlorobenzene or mixtures thereof.

A further preferred reaction medium for the condensation is glacial acetic acid, in which case the reaction is preferably carried out at the boiling temperature of the mixture under reflux. The condensation can also be carried out in aqueous hydrochloric acid or in aqueous sulfuric acid in the presence or absence of an auxiliary such as for example a dispersant or a wetting agent.

The compounds of formula (VIII) are known and obtainable by the following methods known in the literature. They are also commercially available, for example from Sigma-Aldrich Inc., Merck KGaA, Chempur Feinchemikalien and Forschungsbedarf GmbH or Acros Organics.

The dyes of formula (I) according to the present invention can be used directly for polymer coloration, or they are subjected to a finishing (conditioning) operation to convert them into a salable dye preparation.

Finishing can be effected by proceeding from a single dye of formula (I) or from a mixture of two or more dyes of formula (I) or mixtures of one or more of the dyes of formula (I) and dyes of other dye classes, for example pigments or solvent dyes, if appropriate with the assistance of auxiliaries, for example surface modifiers and dispersants, by dispersing, suspending or dissolving in a liquid or solid carrier material and also, if appropriate, standardizing to a desired color strength and hue and, if appropriate, drying the preparation thus obtained.

By adding pigments insoluble in polymers, titanium dioxide is an example, it is possible to obtain corresponding valuable hiding colorations. Titanium dioxide can be added in an amount of 0.01% to 10% by weight and preferably 0.1% to 5% by weight, based on the amount of polymer.

Preparations comprising dyes of formula (I) may further comprise auxiliaries for modifying viscosity/flowability.

Useful auxiliaries of this kind are described for example in U.S. Pat. No. 6,605,126. Preferred examples are ethylene glycols, propylene glycols, polyether polyols, polyester polyols, lactones and carbonic esters.

The present invention accordingly also provides dye preparations comprising one or more dyes of formula (I) and also one or more auxiliaries for modifying viscosity/flowability.

These dye preparations preferably contain one or more dyes of formula (I) in amounts of 5% to 100% by weight and one or more auxiliaries for modifying viscosity/flowability in amounts of 0% to 95% by weight, all based on the dye preparation.

The present invention further provides for the use of the dyes of formula (I) for coloring a polymer. Suitable polymers are for example polyolefins, polyurethane, thermoplastic polyurethane, polyvinyl chloride, polyesters, polyamides, polycarbonates, polystyrene, and also silicones and thermoplastic silicones. Preferred polymers are polyolefins, for example polyethylene or polypropylene and copolymers with polyolefins.

A possible procedure in the use according to the present invention is for the dye of formula (I) to be admixed to the polymer.

In addition, dyes of formula (I) according to the present invention can also be used in the form of masterbatches. Masterbatches are dye concentrates consisting of carrier materials and colorants, the colorants being present in higher concentration than in the final use and the carrier materials being constituted such that they have compatibility with the materials to be colored. The carrier materials used can be polymers, for example polyolefins, polyurethane, thermoplastic polyurethane, polyvinyl chloride, polyesters, polyamides, polycarbonates or polystyrene or else silicones or thermoplastic silicones. Preferred polymers are polyolefins, for example polyethylene or polypropylene and copolymers with polyolefins. Useful carrier materials further include paraffin oils and polyglycols. The dye masterbatches are characterized in particular in that they contain one or more dyes of formula (I) according to the present invention in amounts of 5% to 60% by weight and one or more carrier materials in amounts of 40% to 95% by weight.

The dyes of formula (I) have advantages in bleed/migration fastness in polyolefin mass coloration in particular, compared with commercially available solvent dyes. These advantages are particularly noticeable in the coloration of polypropylene, polypropylene-co-polymers and polypropylene blends.

The examples hereinbelow serve to elucidate the invention without restricting the invention to these examples. Parts and percentages are by weight, unless otherwise stated. Parts by weight relate to parts by volume as the kilogram relates to the liter.

### EXAMPLE 1

a) A mixture of 31.4 parts of the compound of formula (IXa)

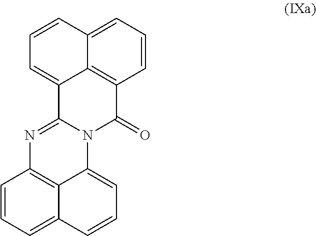

and 93 parts of chlorosulfonic acid is stirred at 60° C. for 2 hours. The reaction mixture is cooled down and then admixed with 31 parts of thionyl chloride, stirred overnight and subsequently poured onto ice-water. The isolated precipitate is washed neutral with water and dried to leave 47.5 parts of a mixture of dyes of formula (VIIa)

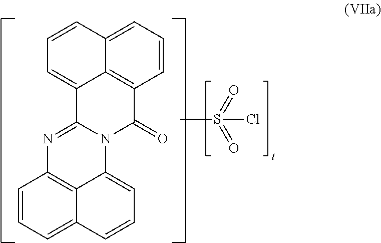

where t is 1 or 2.

a) A mixture of 80 parts of aqueous dioxane, 13.1 parts of hexadecylamine (from Merck KGaA), 4.15 parts of sodium carbonate and 10.74 parts of the compound of formula (VIIa) is stirred at 50° C. for 5 hours. After the reaction has ended, the reaction mixture is admixed with methanol. The isolated precipitate is purified and dried to leave 15 parts of a mixture of dyes of formula (Ig)

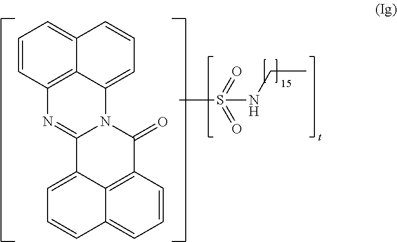

where t is 1 or 2.
b) 1 g of the dye obtained as per a) is comminuted in a mortar and added to altogether 2 kg of polypropylene pellet (Moplen RP340R from Basell). This mixture is mulled on a roller mill until homogeneous and then extruded in a twin-screw extruder (ZSE 18HP-D40 from Leistritz) and pelletized. The pellet obtained can be processed in an injection-molding machine (420 C 1000-100 from Arburg) to form transparent orange-red sample plaques. The dye has high bleed fastness as per prEN14469-4, a high color stability to heat as per EN12877-2 and high lightfastness as per EN ISO 105-B01.

### EXAMPLE 2

a) A mixture of 80 parts of aqueous dioxane, 13.5 parts of octadecylamine (from Chempur Feinchemikalien and Forschungsbedarf GmbH), 4.15 parts of sodium carbonate and 10.70 parts of the compound of formula (VIIa) is stirred at 50° C. for 5 hours. After the reaction has ended, the reaction mixture is admixed with methanol. The isolated precipitate is purified and dried to leave 15.1 parts of a mixture of dyes of formula (Ih)

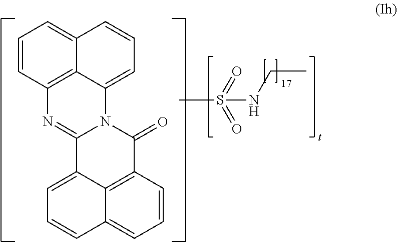

where t is 1 or 2.
b) 1.28 g of the dye obtained as per a) are comminuted in a mortar and added to altogether 2 kg of polypropylene pellet (Moplen RP340R from Basell). This mixture is mulled on a roller mill until homogeneous and then extruded in a twin-screw extruder (ZSE 18HP-D40 from Leistritz) and pelletized. The pellet obtained can be processed in an injection-molding machine (420 C 1000-100 from Arburg) to form transparent orange-red sample plaques. The dye has high bleed fastness as per prEN14469-4, a high color stability to heat as per EN12877-2 and high lightfastness as per EN ISO 105-B01.

### EXAMPLE 3

a) A mixture of 40 parts of aqueous dioxane, 9.3 parts of dodecylamine (from Sigma-Aldrich Inc.), 4.15 parts of sodium carbonate and 10.74 parts of the compound of formula (VIIa) is stirred at 50° C. for 5 hours. After the reaction has ended, the reaction mixture is admixed with methanol. The isolated precipitate is purified and dried to leave 6.9 parts of a mixture of dyes of formula (II)

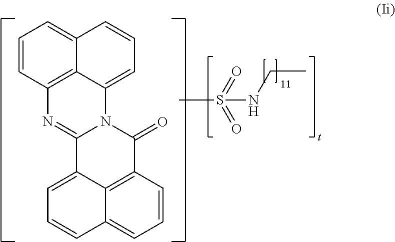

where t is 1 or 2.
b) 1 g of the dye obtained as per a) is comminuted in a mortar and added to altogether 2 kg of polypropylene pellet (Moplen RP340R from Basell). This mixture is mulled on a roller mill until homogeneous and then extruded in a twin-screw extruder (ZSE 18HP-D40 from Leistritz) and pelletized. The pellet obtained can be processed in an injection-molding machine (420 C 1000-100 from Arburg) to form transparent orange-red sample plaques. The dye has high bleed fastness as per prEN14469-4, a high color stability to heat as per EN12877-2 and high lightfastness as per EN ISO 105-B01.

Similarly to the procedures described in examples 1 to 3 it is also possible to prepare the inventive dyes of formulae (Ia1) to (Ia41), (Ib1) to (Ib29), (Ic1) to (Ic29), (Id1) to (Id37), (Ie1) to (Ie26) and (If1) to (If30). They likewise have high bleed fastnesses as per prEN14469-4, a high color stability to heat as per EN12877-2 and high lightfastnesses as per EN ISO 105-B01.

### Comparative Example 1

1 g of the dye of structure (IXa) is comminuted in a mortar and added to altogether 2 kg of polypropylene pellet (Moplen RP340R from Basell). This mixture is mulled on a roller mill until homogeneous and then extruded in a twin-screw extruder (ZSE 18HP-D40 from Leistritz) and pelletized. The pellet obtained can be processed in an injection-molding machine (420 C 1000-100 from Arburg) to form transparent red sample plaques.

The bleed fastness as per prEN14469-4 of the product thus obtained and also of the products obtained according to examples 1, 2 and 3 was determined with the following result:

| | Example | | | |
|---|---|---|---|---|
| | Example | Example | Example | Comparative |
| | 1 | 2 | 3 | example 1 |
| Bleed fastness | 4-5 | 4-5 | 4 | 1 |

1-11. (canceled)

12. A dye of formula (I) 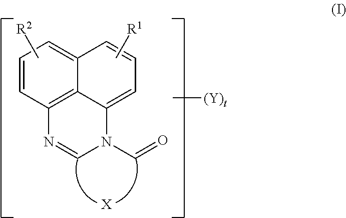
where X represents a group of formula (II) or of formula (III) 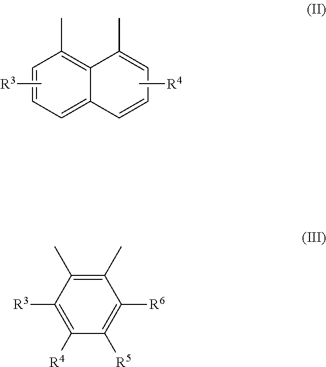
Y represents a group of formula (IV) 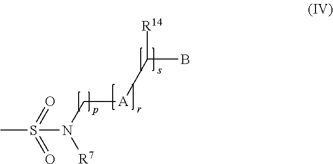
R¹ to R⁶ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, N-mono-(C₁-C₂₂)-alkylcarbamoyl, N,N-di-(C₁-C₂₂)-alkylcarbamoyl, (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl, mono-(C₁-C₂₂)-alkylaminosulfonyl, di-(C₁-C₂₂)-alkylaminosulfonyl or arylsulfonyl;
R⁷ represents hydrogen, (C₁-C₂₂)-alkyl or phenyl-(C₁-C₂₂)-alkyl;
R¹⁴ represents hydrogen or (C₁-C₆)-alkyl;
A represents a group of formula (V) or of formula (VI) 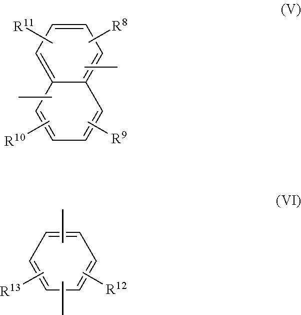
wherein
R⁸ to R¹³ each independently represent hydrogen, (C₁-C₁₂)-alkyl, trifluoromethyl, halogen, cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio, (C₁-C₆)-alkylsulfonyl, vinyl, hydroxyethyl, mono-(C₁-C₂₂)-alkylaminoethylenesulfonyl, di-(C₁-C₂₂)-alkylaminoethylenesulfonyl, mono-(C₁-C₂₂)-alkylaminosulfonyl or di-(C₁-C₂₂)-alkylaminosulfonyl wherein in each case the (C₁-C₂₂)-alkyl radical may be interrupted by one or more oxygen atoms and/or contain one or more hydroxyl groups;
B represents hydrogen or methyl;
p and s are each independently a number from 0 to 22;
r is 0 or 1; and
t is a number from 1 to 6;
wherein Y is attached to the naphthalene ring bearing R¹ and R², to the group X and/or to an aryl radical which is a constituent part of the R¹ to R⁶ groups, and Y attached to the group of formula (III) represents R³, R⁴, R⁵ and/or R⁶; and
r, s, p and R¹⁴ have identical or different meanings within a molecule of formula (I); and
s is a number from 2 to 22 when X represents a group of formula (III), t is 1, R⁷ represents hydrogen, p is 0 and r is 1; and
excluding dyes of formula (I)
where X represents a group of formula (II), R¹ and R² each represent hydrogen, R³ and R⁴ each represent hydroxyl, t is 1, p and s are each 0, r is 1, A represents a group of formula (VI) and R⁷, R¹², R¹³ and B each represent hydrogen; and
where X represents a group of formula (III), R¹ to R⁶ each represent hydrogen, t is 1 and Y represents —SO₂N((C₁-C₅)-alkyl)₂.

13. The dye of formula (I) as claimed in claim 12, conforming to one of formulae (Ia) to (If) 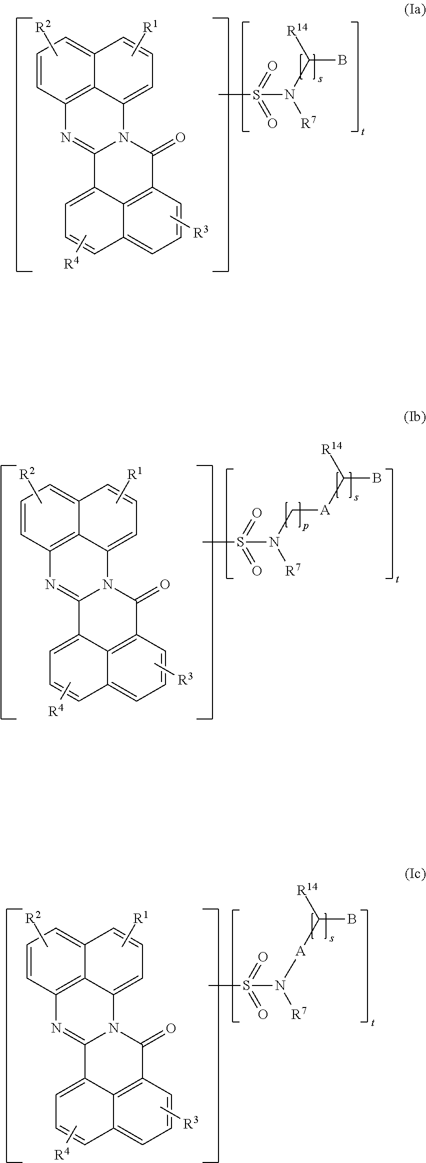 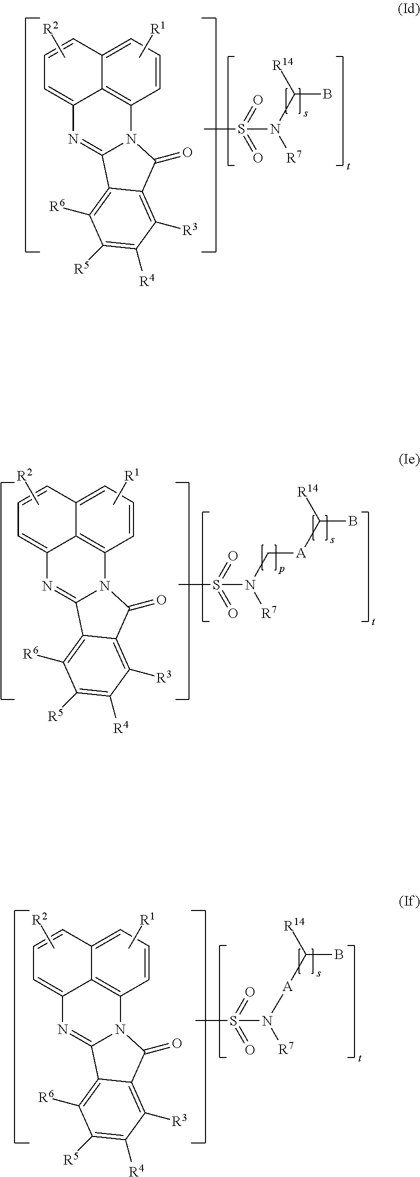
where R¹ to R⁷, R¹⁴, A, B, s and t are each as defined in claim 12 and p is a number from 1 to 22 in formulae (Ib) and (Ie) and is as defined in claim 12 in formulae (Ia), (Ic), (Id) and (If).

14. The dye of formula (I) as claimed in claim 13, conforming to formula (Ia) wherein R¹ to R⁴ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl, mono-(C₁-C₂₂)-alkylaminosulfonyl, di-(C₁-C₂₂)-alkylaminosulfonyl or arylsulfonyl;
R⁷ represents hydrogen or (C₁-C₂₂)-alkyl; and
R¹⁴, B, s and t are previously defined in claim 13.

15. The dye of formula (I) as claimed in claim 13, conforming to formula (Ib) wherein R¹ to R⁴ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl, mono-(C₁-C₂₂)-alkylaminosulfonyl, di-(C₁-C₂₂)-alkylaminosulfonyl or arylsulfonyl;
A represents a group of formula (V) or of formula (VI) where
R⁸ to R¹³ each independently represent hydrogen, (C₁-C₁₂)-alkyl, halogen, cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio, (C₁-C₆)-alkylsulfonyl or mono-(C₁-C₂₂)-alkylaminosulfonyl or di-(C₁-C₂₂)-alkylaminosulfonyl wherein the alkyl radical may be interrupted by one or more oxygen atoms and/or contain one or more hydroxyl groups;
p is a number from 1 to 4;
s is a number from 0 to 6; and
B, R⁷, R¹⁴ and t are each as defined in claim 13.

16. The dye of formula (I) as claimed in claim 13, conforming to formula (Ic) wherein R¹ to R⁴ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl, mono-(C₁-C₂₂)-alkylaminosulfonyl, di-(C₁-C₂₂)-alkylaminosulfonyl or arylsulfonyl;
A represents a group of formula (V) or of formula (VI) where
R⁸ to R¹³ each independently represent hydrogen, (C₁-C₁₂)-alkyl, halogen, cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio, (C₁-C₆)-alkylsulfonyl or mono-(C₁-C₂₂)-alkylaminoethylenesulfonyl, di-(C₁-C₂₂)-alkylaminoethylenesulfonyl, mono-(C₁-C₂₂)-alkylaminosulfonyl or di-(C₁-C₂₂)-alkylaminosulfonyl wherein the (C₁-C₂₂)-alkyl radical may in each case be interrupted by one or more oxygen atoms and/or contain one or more hydroxyl groups;
R⁷ represents hydrogen, (C₁-C₁₈)-alkyl or phenyl-(C₁-C₄)-alkylene; and
B, R¹⁴, s and t are each as defined in claim 13.

17. The dye of formula (I) as claimed in claim 13, conforming to formula (Id) wherein R¹ to R⁶ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, N-mono-(C₁-C₂₂)-alkylcarbamoyl, N,N-di-(C₁-C₂₂)-alkylcarbamoyl, (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl or arylsulfonyl;
R⁷ represents hydrogen or (C₁-C₂₂)-alkyl; and
B, R¹⁴, s and t are each as defined in claim 13.

18. The dye of formula (I) as claimed in claim 13, conforming to formula (Ie) wherein R¹ to R⁶ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, N-mono-(C₁-C₂₂)-alkylcarbamoyl, N,N-di-(C₁-C₂₂)-alkylcarbamoyl, (C₁-C₆)-alkoxycarbonyl; aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl or arylsulfonyl;
A represents a group of formula (V) or of formula (VI) where
R⁸ to R¹³ each independently represent hydrogen, (C₁-C₁₂)-alkyl, halogen, cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio, (C₁-C₆)-alkylsulfonyl or mono-(C₁-C₂₂)-alkylaminosulfonyl or di-(C₁-C₂₂)-alkylaminosulfonyl wherein the alkyl radical may be interrupted by one or more oxygen atoms and/or contain one or more hydroxyl groups;
R⁷ represents hydrogen, (C₁-C₁₈)-alkyl or phenyl-(C₁-C₆)-alkylene;
p is a number from 1 to 4;
s is a number from 0 to 6; and
B, R¹⁴ and t are each as defined in claim 13.

19. The dye of formula (I) as claimed in claim 13, conforming to formula (If) wherein R¹ to R⁶ each independently represent hydrogen, (C₁-C₆)-alkyl, trifluoromethyl, cyclo-(C₃-C₈)-alkyl, aryl, halogen, cyano, nitro, hydroxyl, (C₁-C₆)-alkoxy, aryloxy, (C₂-C₆)-acyl, arylcarbonyl, (C₂-C₆)-acyloxy, arylcarbonyloxy, (C₂-C₆)-acylamino, arylcarbonylamino, carbamoyl, (C₁-C₆)-alkoxycarbonyl, aryloxycarbonyl, amino, monocyclo-(C₃-C₈)-alkylamino, mono-(C₁-C₆)-alkylamino, di(cyclo)-(C₃-C₈)-alkylamino, di-(C₁-C₆)-alkylamino, monoarylamino, diarylamino, mono-(C₁-C₆)-alkylmonoarylamino, aminothiocarbonylamino, aminocarbonylamino, aminosulfonylamino, (C₁-C₆)-alkylthio, arylthio, (C₁-C₆)-alkylsulfonyl, aryloxysulfonyl, mono-(C₁-C₂₂)-alkylaminosulfonyl, di-(C₁-C₂₂)-alkylaminosulfonyl or arylsulfonyl;
A represents a group of formula (V) or of formula (VI) where
R⁸ to R¹³ each independently represent hydrogen, (C₁-C₁₂)-alkyl, halogen; cyano, nitro, hydroxyl, (C₁-C₆)-alkoxy, (C₂-C₆)-acyl, (C₂-C₆)-acylamino, carbamoyl, amino, mono-(C₁-C₁₂)-alkylamino, (C₁-C₆)-alkylthio, (C₁-C₆)-alkylsulfonyl or mono-(C₁-C₂₂)-alkylaminoethylenesulfonyl, di-(C₁-C₂₂)-alkylaminoethylenesulfonyl, mono-(C₁-C₂₂)-alkylaminosulfonyl or di-(C₁-C₂₂)-alkylaminosulfonyl wherein the (C₁-C₂₂)-alkyl radical may in each case be interrupted by one or more oxygen atoms and/or contain one or more hydroxyl groups;
R⁷ represents hydrogen, (C₁-C₁₈)-alkyl or phenyl-(C₁-C₄)-alkylene; and
B, R¹⁴, s and t are each as defined in claim 13.

20. A process for preparing the dye of formula (I) as claimed in claim 12, which comprises reacting a compound of formula (VII) 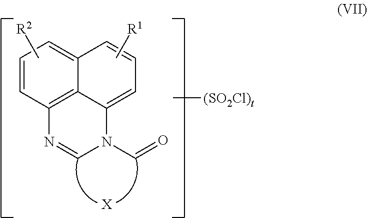
where R¹, R², X and t are each as defined in claim 12, with a compound of formula (VIII) 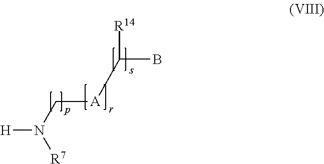
where A, B, R⁷, R¹⁴, p, r and s are each as defined in claim 12.

21. A process for coloring a polymer which comprises utilizing the dye of formula (I) as claimed in claim 12.

22. A masterbatch comprising the dye of formula (I) as claimed in claim 12 and a carrier material.
